(19) United States
(12) Patent Application Publication
(10) Pub. No.: US 2011/0007269 A1
(43) Pub. Date: Jan. 13, 2011

(54) METHOD AND APPARATUS OF MEASURING OPTICAL PARAMETERS OF A PERSON USING A LIGHT FIELD

(76) Inventors: Stephan Trumm, Munich (DE); Rainer Sessner, Roth (DE); Andrea Peters, Munich (DE); Leonhard Schmid, Raisting (DE); Dietmar Uttenweiler, Icking (DE); Jochen Brosig, Anzing (DE)

Correspondence Address: DICKSTEIN SHAPIRO LLP, 1633 Broadway, NEW YORK, NY, 10019, US

(21) Appl. No.: 12/812,153

(22) PCT Filed: Nov. 18, 2008
(86) PCT No.: PCT/EP08/09741

§ 371 Date: Sep. 28, 2010

(30) Foreign Application Priority Data
Jan. 10, 2008 (DE) 10 2008 003 906.3

(51) Int. Cl. A61B 3/14

(52) U.S. Cl. 351/204; 351/206; 351/246

(57) ABSTRACT

A method and apparatus provided to measure optical parameters of a person wearing spectacles. One or more fixation targets are provided to generate a flat extensive light field that can align the direction of sight of the person when the person looks at the light filed. Image recording devices are provided to generate image data of subareas of the person's head and a data processing unit can determine the optical parameters based on the generated image data.

### BACKGROUND

The preferred embodiments described herein relate to a use of at least one fixation target, and to an apparatus.

Due to the introduction of individually optimized spectacle lenses, it is possible to aid the needs of persons having visual defects and, for example, to provide spectacle lenses having individually optimized viewing zones. Custom-fitted spectacle lenses enable an optimal correction of optical visual defects of a wearer of the spectacle lenses. Individual calculation and fitting of spectacle lenses is also possible for sports spectacles, which are distinguished by strong bending, face form and pantoscopic angles.

In order to fully utilize the optical advantages of individual spectacle lenses, and in particular, of individually fitted progressive lenses, it is necessary to calculate and produce these spectacle lenses taking into account information such as the user's position of wear, and to accordingly wear them according to the position of wear used for calculation and production. Furthermore, the position of wear depends on a multitude of parameters including, for example, the interpupillary distance of the user, the face form angle, the spectacle lens pantoscopic angle, the spectacle frame, the corneal vertex distance of the system of lens and eye, the fitting height of the spectacle lenses and the like. These and further parameters, which may be taken into account or are necessary for describing the position of wear, are provided in relevant standards, such as DIN EN ISO 1366, DIN 58 208, DIN EN ISO 8624, and DIN 5340. Furthermore, it is necessary to arrange or center the spectacle lenses in a spectacle frame according to the optical parameters used for the production, so that the spectacle lenses are indeed worn in the position of wear according to the optical parameters.

A multitude of measuring instruments is available to the optician for determining the individual optical parameters. With a so-called pupillometer, for example, the optician can analyze pupillary reflexes or determine the distance of the pupil centers to thus obtain the interpupillary distance, such that an LED is mapped to infinity, for example.

Pantoscopic angle and the corneal vertex distance may be determined with a measuring instrument in which, in the customer's habitual head and body posture, the measuring instrument is held on a frame plane of a spectacle frame. The pantoscopic angle may be read off laterally via a gravity-driven pointer on the basis of a scale. An engraved ruler is used for determining the corneal vertex distance, with which the distance between the estimated groove bottom of the spectacle frame and the cornea is also measured from the side.

The face form angle of the spectacle frame may be determined with a measuring instrument on which the spectacles are placed. The nasal rim of a lens or spectacle lens shape has to be arranged over a center of rotation of a movable measuring arm, wherein the other lens or spectacle lens shape is parallel to an engraved line. The measuring arm is adjusted such that a marked axis of the measuring arm is parallel to the frame plane of the lens arranged thereabove. Subsequently, the face form angle can be read off a scale.

Moreover, there is the possibility of locating the view of a test person by having the test person focus his root of the nose in a mirror image. It is also possible to use a speckle pattern or a luminous point.

All above-mentioned possibilities have the object of aligning the view of the person (hereinafter referred to as “test person”) to measure the optical parameters such that the actual alignment of the pupils corresponds to the viewing behavior to be measured.

The preferred embodiments enable the optical parameters of a test person to be measured substantially corresponding to his natural viewing behavior.

### BRIEF DESCRIPTION OF THE DRAWINGS

Preferred embodiments will be described in the following on the basis of accompanying figures in which:

FIG. 1 shows a perspective schematic view of an apparatus in an operating position in accordance with an exemplary embodiment

FIG. 2 shows a schematic sectional plan view of an arrangement of the image recording devices according to FIG. 1 in an operating position in accordance with an exemplary embodiment

FIG. 3 shows a schematic sectional side view of an arrangement of the image recording devices according to FIG. 1 in an operating position in accordance with an exemplary embodiment

FIG. 4 shows a schematic sectional plan view of a further embodiment in an operating position in accordance with an exemplary embodiment

FIG. 5 shows a schematic view of exemplary image data in accordance with an exemplary embodiment

FIG. 5a shows a schematic view of exemplary image data in accordance with an exemplary embodiment

FIG. 5b shows a schematic view of exemplary image data in accordance with an exemplary embodiment

FIG. 6 shows a further schematic view of exemplary image data in accordance with an exemplary embodiment

FIG. 6a shows a further schematic view of exemplary image data in accordance with an exemplary embodiment

FIG. 6b shows a further schematic view of exemplary image data in accordance with an exemplary embodiment

FIG. 7 shows exemplary image data according to FIG. 5 in accordance with an exemplary embodiment

FIG. 7a shows a schematic view of exemplary comparative image data in accordance with an exemplary embodiment

FIG. 7b shows exemplary image data according to FIG. 5b in accordance with an exemplary embodiment

FIG. 8 shows exemplary image data according to FIG. 6 in accordance with an exemplary embodiment

FIG. 8a shows exemplary image data according to FIG. 6b in accordance with an exemplary embodiment

FIG. 9 shows exemplary output data as output according to one embodiment in accordance with an exemplary embodiment

FIG. 9a shows exemplary output data in accordance with an exemplary embodiment

FIG. 10 shows a front view of a section of an apparatus in accordance with an exemplary embodiment

FIG. 11a shows a top view of a schematic illustration of a fixation target in accordance with an exemplary embodiment

FIG. 11b shows a top view of a schematic illustration of a fixation target in accordance with an exemplary embodiment

FIG. 11c shows a top view of a schematic illustration of a fixation target in accordance with an exemplary embodiment

FIG. 12 shows a lateral sectional view of a schematic illustration of a fixation target in accordance with an exemplary embodiment

FIG. 13 shows a schematic sectional view of an exemplary fixation target in top view in accordance with an exemplary embodiment

FIG. 14 shows a schematic perspective view of two fixation targets in accordance with an exemplary embodiment

FIG. 15 shows a schematic front view of a section of an apparatus in accordance with an exemplary embodiment

FIG. 16 shows a schematic lateral sectional view of a fixation target in accordance with an exemplary embodiment

FIG. 17 shows a schematic sectional top view of a section of an apparatus in accordance with an exemplary embodiment

FIG. 18 shows an enlarged section of FIG. 17 in accordance with an exemplary embodiment

FIG. 19 shows a schematic view of a section of FIG. 17 in accordance with an exemplary embodiment

FIG. 20 shows a perspective schematic view of a component of a fixation target; and

FIG. 21 shows a schematic sectional view of the object of FIG. 20 in accordance with an exemplary embodiment

### DEFINITION OF TERMS

Prior to the following detailed description of the preferred embodiments, terms contributing to the understanding of the preferred embodiments will be defined or described as follows.
An “auxiliary structure” can be an artificial structure arranged, for example, on a head, and preferably on a face. The auxiliary structure can also be the entire face, a part of the face, a part of the head, the shape of the head, the position of characteristic parts of the head or the face, such as the ears, the nose, pigments, a birthmark, freckles, one or both eyebrows, and the like. The auxiliary structure can also comprise one or more adhesive labels stuck on the head or the face.
An “eye corresponding” to a spectacle lens is the eye of a user of the spectacle lens, i.e. the eye of the spectacle wearer, in front of which the spectacle lens is arranged. In other words, the “eye corresponding” to the spectacle lens is the eye of the spectacle wearer with which they look through the spectacle lens. The right eye of the spectacle wearer corresponds to the right spectacle lens and the left eye corresponds to the left spectacle lens. Thus, both eyes correspond to the spectacles of a spectacle wearer.
Spectacle lenses can be single-vision lenses, multifocal lenses, progressive lenses, with or without tint, reflective coating and/or polarization filters, for example.
The term “determining” includes “calculating”, “reading from a table”, “taking from a database”, and the like.
The position of a spectacle lens relative to a pupil center, in particular, includes all information necessary to indicate the arrangement of the spectacle lens relative to the pupil center, such as forward inclination of the spectacle lens, position of a lens plane or spectacle lens shape plane relative to the pupil center and, in particular, also relative to the zero direction of sight, location of optically particularly relevant regions, such as near reference point or zone, distance reference point or zone, etc., position of the centration point, astigmatism axis, and the like.
“Characteristic points” of a spectacle lens are e.g. points making the alignment or the arrangement of the spectacle lens determinable in an unambiguous manner. For example, characteristic points may be engraved points of the spectacle lens or reference points of the spectacle lens. Preferably, characteristic points may be two-dimensional, flat forms, such as circles, crosses, and the like.
“Engraved points” can be such points allowing an unambiguous determination of the optical properties. For example, the relative position of the near reference point, distance reference point, umbilical line, and the like, with respect to a centration point is known as the preferred engraved point. A spectacle lens may have one or more characteristic points, consequently, one or more characteristic points can be presented by the presenting means. Furthermore, engraved points are formed such that they are substantially not visible to the naked eye, i.e. without further optical aids.

For example, engraved points can be two or more product-specific micro engravings, such as circle(s), rhombus(es), etc., which are in particular arranged at a standardized distance from each other, e.g. at a distance of approximately 34 mm. These engraved points are referred to as “main engravings”. Moreover, engraved points, and specifically micro engravings, may define a horizontal axis. The center between the two engraved points is also the point of origin (hereinafter referred to as “zero point”) for the further measuring and reference points if stamped on, lens-specific marks of the spectacle lens are missing.

Directly below the “main engravings”, the engraving of the addition and an index for the base curve and refractive index of the lens may be provided temporally and nasally, respectively.

In addition, a further engraved point may be a trademark, for example in the form of a letter, etc., which may be disposed approximately 13 mm below the “main engraving” or the engraving of the addition and the index for the base curve and the refractive index of the lens.
A “presenting means” may be an adhesive label, a point, and preferably a drawn point or circle or another two-dimensional object and/or a three-dimensional object. A presenting means may also comprise several adhesive labels and/or points, preferably drawn points or circles or other two-dimensional objects and/or three-dimensional objects. A presenting means differs from an auxiliary structure in that the presenting means is associated with a spectacle lens, for example, by the presenting means comprising an adhesive label stuck on the spectacle lens. The auxiliary structure is associated with the head or the face of a user, for example, by the auxiliary structure comprising an adhesive label stuck on the face.

Moreover, a spectacle lens may have one or more characteristic points that can be presented by one or more presenting means. For example, one or more engraved points can be presented by one or more presenting means. The presenting means can be an adhesive label arranged such that the position of one or more engraved points relative to the adhesive label can be unambiguously determined. Further more, an adhesive label may cover two (or more) engraved points, and the adhesive label may be colored at the positions overlapping the engraved points, wherein the color differs from the remaining color of the adhesive label. For example, the adhesive label may have a white base color or be transparent, and at positions overlapping the two (or three) engraved points the adhesive label may have at least one black point or circle or two (or three) saddle points.

Furthermore, a presenting means can preferably comprise one or more stamped-on markings, such as two stamped on arcs of the form “( )”, in the middle of which the distance reference point B_{F} of a spectacle lens can be located. The arcs can be arranged such that the distance reference point is approximately 8 mm above the zero point (see above). Two horizontal lines on the left and right thereof are auxiliary markings for aligning the lens horizontal when checking the cylinder axis.

Moreover, a stamped-on marking may comprise a distance centration cross arranged approximately 4 mm above the zero point (see above) for example. The distance centration cross is the fitting cross for the exact centration of the lens in front of the eye or the frame.
The “lens horizontal” (see above) may comprise two horizontal, interrupted lines temporally/nasally each. Preferably, a specific product engraving in the form of one or more circles, rhombuses or the like is arranged between the lines.

In addition, a stamped-on marking may comprise a prism reference point B_{P} preferably coinciding with the zero point (see above).

The stamped-on marking may also comprise a circle around the near reference point B_{N}. In the exemplary embodiment, the near reference point, i.e., the center of the circle, may be displaced downwardly and nasally from the zero point by approximately 14 mm and approximately 25 mm, respectively. This is an exemplary auxiliary measuring point in order to be able to test the near power on the focimeter (also referred to as “SBM”). The real lateral displacement of the near visual point may deviate therefrom depending on the variable inset.

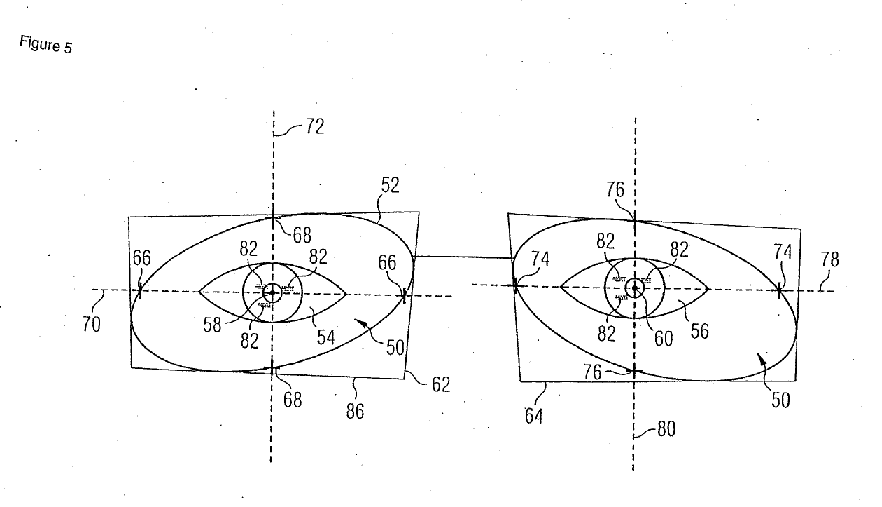

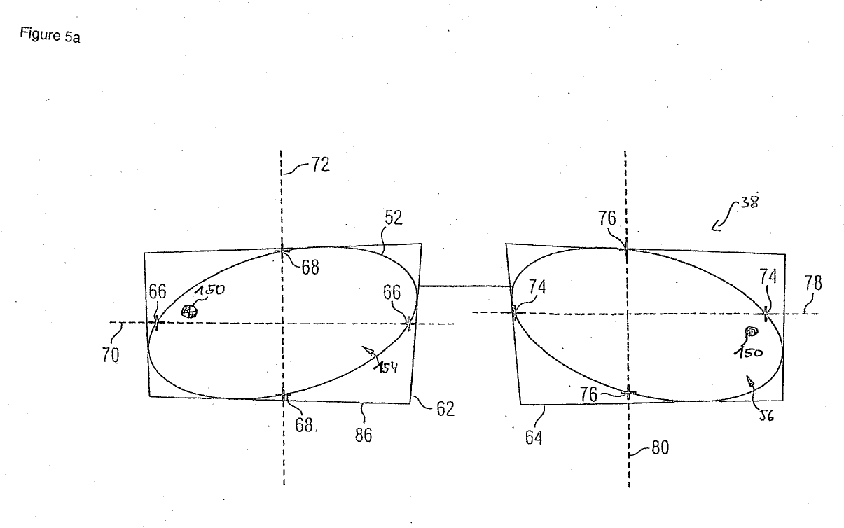

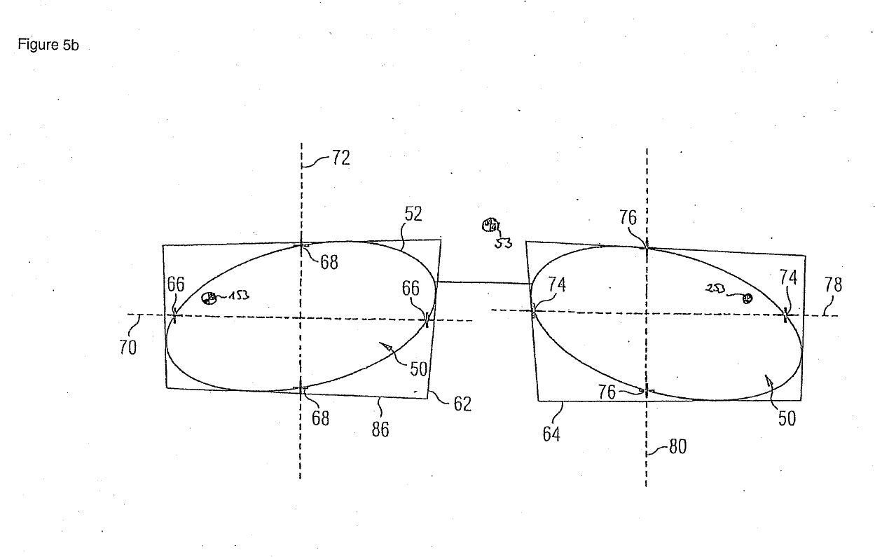

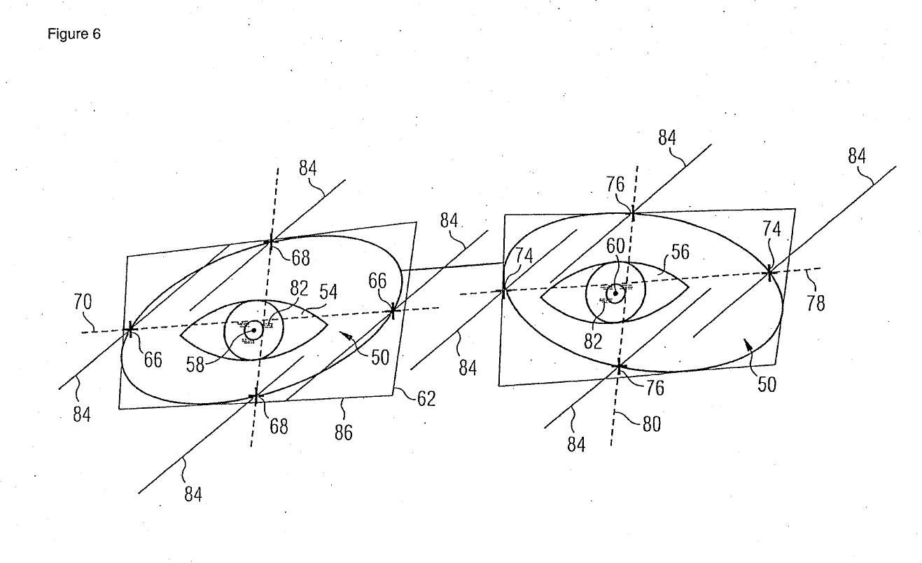

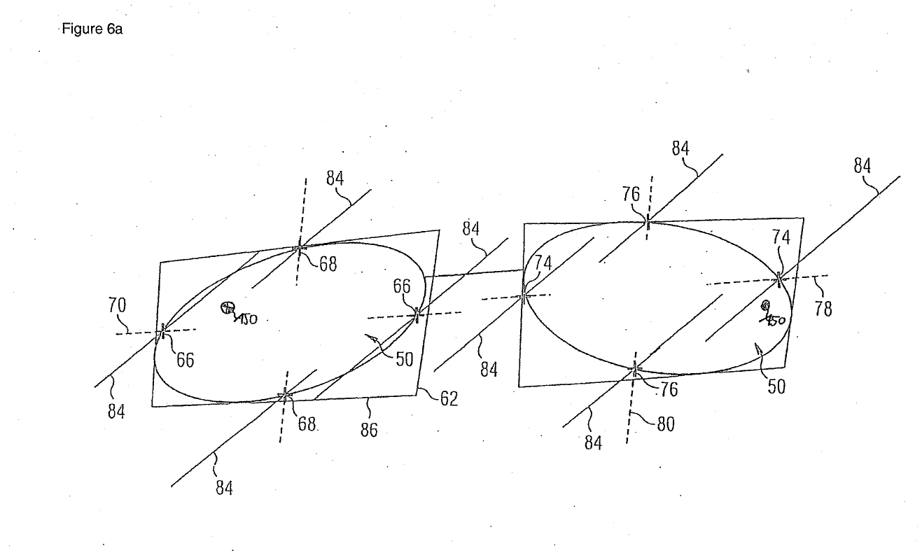

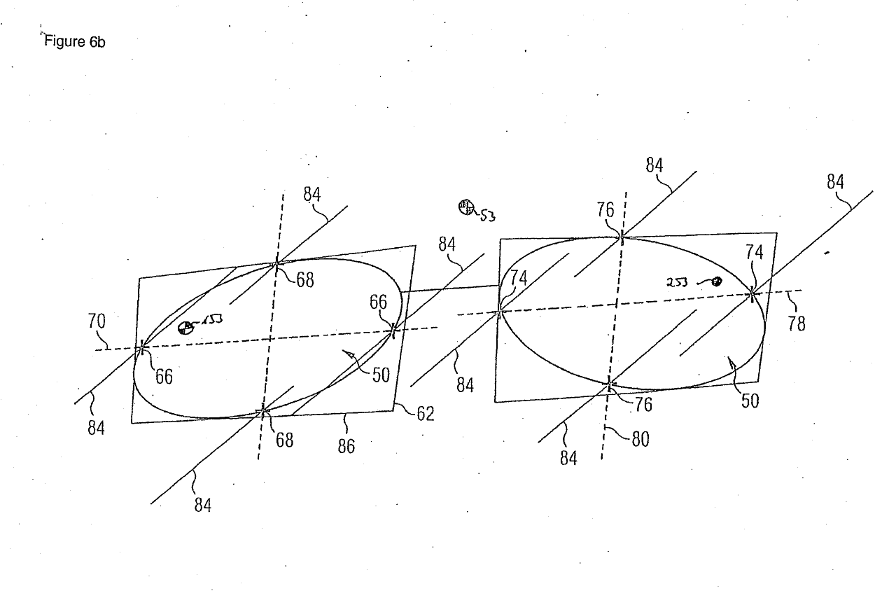

Furthermore, the stamped-on markings may have further or additional markings, for example, a schematic eye to mark in particular the distance reference point, plus and minus signs, points to indicate the near reference point, and the like.
Two “image recording devices” are for example two digital cameras, which are positioned separately from each other. It is possible that an image recording device preferably comprises a digital camera and at least one optical deflecting element or mirror, wherein image data of a subarea of a head can be recorded or generated with the camera by means of the deflecting minor. Therefore, two image recording devices likewise comprise two digital cameras and at least two deflecting elements or mirrors, wherein each digital camera and at least one deflecting minor constitute an image recording device. Further preferably, two image recording devices may also consist of exactly one digital camera and two deflecting elements or minors, wherein image data are recorded or generated by means of the digital camera in a time-shifted manner. For example, image data are generated at a first point of time, wherein a subarea of a head is imaged by means of said one deflecting mirror, and image data are generated at a second point of time, which image data image the subarea of the head by means of the other deflecting minor. Furthermore, the camera may also be arranged such that image data are generated by the camera at the first or the second point of time, wherein no deflecting mirror is necessary or arranged between the camera and the head. In the exemplary embodiment, the two image recording devices can generate image data under different recording directions.
Two different “recording directions” mean that different image data are generated for overlapping subareas of the head, preferably of one and the same subarea of the head, and in particular, that image data or comparative image data of identical subareas of the head of the user are generated under different perspective views. Consequently, the same subarea of the head is imaged, but the image data or comparative image data differ. Different recording directions can be achieved, for example, in that the image data are generated by at least two image recording devices, wherein effective optical axes of the at least two image recording devices are not in parallel.
Dimensioning in the boxing system is understood in the meaning of the measuring system described in relevant standards, such as in DIN EN ISO 8624 and/or DIN EN ISO 1366 DIN and/or DIN 58 208 and/or DIN 5340. Furthermore, with respect to the boxing system and further conventional terms and parameters used, reference is made to the book “Die Optik des Auges und der Sehhilfen” by Dr. Roland Enders, 1995 Optische Fachveröffentlichung GmbH, Heidelberg, and to the book “Optik und Technik der Brille” by Heinz Diepes and Ralf Blendowske, 2002 Verlag Optische Fachveröffentlichungen GmbH, Heidelberg. Likewise, reference is also made to the brochure“inform fachbereatung für die augenoptik” PR series of texts of the German Optometrists' Association ZVA, issue 9, “Brillenzentrierung”, ISBN 3-922269-23-0, 1998, in which the boxing system is exemplarily illustrated in particular in FIGS. 5 and 6. Moreover, reference is also made to the book “Brillenanpassung Ein Schulbuch und Leitfaden” by Wolfgang Schulz and Johannes Eber 1997, DOZ Verlag, published by the German Optometrists' Association, Düsseldorf, ISBN 3-922269-21-4, in particular to items 1.3, 1.4 and 1.5 and the corresponding figures. The foregoing reference are cited by this disclosure and incorporated by reference.

The delimitation according to dimensioning in the boxing system, for example, comprises frame points for an eye or both eyes, which lie furthest to the outside or inside and/or up or down. These frame points are conventionally determined by means of tangents on the frame or the regions of the spectacle frame assigned to the respective eyes. Refer to standard DIN 58 208, image 3, for reference.

In particular, the boxing system is a rectangle in the plane of lens or plane of spectacle lens shape, which defines a spectacle lens. According to the above-mentioned standards, to determine the plane of lens or plane of spectacle lens shape, one starts from a plane with the normal vector of the cross product of center parallel line/center horizontal line of the box. Generally, the normal of the plane of lens or plane of spectacle lens shape can be determined from the cross product of the vector between the nasal point and the temporal point as well as the vector between the upper and the lower point of the lens rim to the frame. Advantageously, the forward inclination and the face form angle correspond best to the visual situation.
The “retaining point” for the plane of lens or plane of spectacle lens shape is approximated as follows:

The starting point is the center of the vector between the upper and the lower point. Subsequently, it is followed horizontally along the vector between the nasal point and the temporal point in the center of the lens or center of the spectacle lens shape (approximated by the x coordinate). The cross product of the vector between the centers of the planes of lens or planes of spectacle lens shape of both sides and the mean value of the two vectors of upper and lower frame points determines the normal of the frame plane. The retaining point is one of the lens centers or spectacle lens shape centers.

The boxing system is determined as a perpendicular projection of the lens rim or spectacle lens shape rim to the plane of lens or plane of spectacle lens shape. Next, the face form angle can be determined for each side as the angle between the respective plane of lens or plane of spectacle lens shape and the frame plane.

In other words, the normal of the plane of lens or plane of spectacle lens shape can be determined from the cross product of the vector between the nasal and the temporal intersection point of a horizontal plane through the straight line of the zero direction of sight with the respective lens rim to the frame as well as the vector between the upper and the lower intersection point of a vertical plane through the straight line of the zero direction of sight with the respective lens rim to the frame.
The “interpupillary distance” substantially corresponds to the distance of the pupil centers, preferably in the zero direction of sight.
The “zero direction of sight” is a direction of sight straight ahead with parallel fixing lines. In other words, it is a direction of sight defined by a position of the eye relative to the head of the user, wherein the eyes look at an object that is at eye level and is arranged at an infinitely distant point. Consequently, the zero direction of sight is merely determined by the position of the eyes relative to the head of the user. If the head of the user is in a normal upright posture, then the zero direction of sight substantially corresponds to the horizontal direction in the frame of reference of the earth. However, the zero direction of sight may be tilted with respect to the horizontal direction in the frame of reference of the earth if the user, for example, inclines his head forward or to the side without further movement of the eyes. Analogously, the zero direction of sight of both eyes spans a plane substantially parallel to the horizontal plane in the frame of reference of the earth. The plane, which is spanned by the two zero directions of sight of the two eyes, can also be inclined with respect to the horizontal plane in the frame of reference of the earth if the user inclines his head forward or to the side, for example.

Preferably, the horizontal plane of the user corresponds to a first plane, and the vertical plane of the user corresponds to a second plane perpendicular to the first plane. For example, the horizontal plane in the frame of reference of the user can be arranged in parallel to a horizontal plane in the frame of reference of the earth and merely pass through the center of a pupil. More particularly, this is the case if the two eyes of the user are arranged at different heights (in the frame of reference of the earth), for example.
The ocular center of rotation of an eye is the point of the eye that substantially remains still during a movement of the eye, with a specified head posture, for example, an infraduction or a supraduction by rotation of the eye. Thus, the ocular center of rotation substantially is the rotational center of the eye.
Effective optical axes of the image recording devices are the areas of lines starting from the center of the respective apertures of the image recording devices perpendicularly to these apertures and intersecting the imaged subarea of the head of the user. In other words, the effective optical axes are preferably the optical axes of the image recording devices, wherein these optical axes are conventionally arranged perpendicularly to a lens system of the image recording devices and start from the center of the lens system. If no further optical elements, such as deflecting mirrors or prisms, are present in the ray path of the image recording devices, then the effective optical axis substantially corresponds to the optical axis of the image recording device. However, if further optical elements, one or more deflecting mirrors, for example, are arranged in the ray path of the image recording device, the effective optical axis no longer corresponds to the optical axis as starts from the image recording device.

Put differently, the effective optical axis is the area of an optionally multiple times deflected optical axis of an image recording device which intersects the head of the user without change of direction. The optical axis of the image recording device corresponds to a line starting from a center of an aperture of the image recording device at a right angle with respect to a plane comprising the aperture of the image recording device, wherein the direction of the optical axis of the image recording device can be changed by optical elements, such as mirrors and/or prisms. The effective optical axes of two image recording devices may almost intersect.
The term “almost intersect” means that the effective optical axes have a small distance of less than approximately 10 cm, preferably less than approximately 5 cm, and even more preferably less than approximately 1 cm. Thus, at least almost intersect means that the effective axis intersect or almost intersect.
A “pattern projection device” is a conventional projector, such as a commercial beamer. The projected pattern data are preferably a stripe pattern or a binary stripe pattern. The pattern data are projected onto at least a subarea of the head of the user, and image data and/or comparative image data thereof are generated by means of the image recording device. The image recording device generates image data and/or comparative image data of the illuminated subarea of the head of the user at a triangulation angle. The triangulation angle corresponds to the angle between an effective optical axis of the image recording device and a projection angle of the pattern projection device. Height differences of the subarea of the head correspond to lateral displacements of the stripes of the stripe pattern as preferred pattern data. Preferably, in the phase-measuring triangulation, the so-called phase-shift method is used, wherein a periodic wave pattern, which is approximately sinusoidal in the intensity distribution, is projected onto the subarea of the head, and the wave pattern is moved stepwise in the projector. During the movement of the wave pattern, image data and/or comparative image data are generated by the intensity distribution (and the subarea of the head) preferably three times during a period. The intensity distribution can be inferred from the generated image data and/or comparative image data, and a phase position of the image points with respect to each other can be determined, wherein points on the surface of the subarea of the head are associated with a specific phase position according to the distance from the image recording device. Moreover, reference is made to the thesis entitled “Phasenmessende Deflektometrie (PMD)—ein hochgenaues Verfahren zur Vermessung von Oberflächen” by Rainer Seβner, March 2000, which is hereby incorporated by reference for further definitions of terms.
A “cylinder lens” is a lens substantially having the shape of a cylinder, i.e., whose curved surfaces are cylinder surfaces. In contrast to a spherical lens focusing light onto one single point, the cylinder lens focuses a light ray along a single axis, the “focal axis” or “focal line”. Mathematically, a cylindrical lens can be described in correspondence with a spherical lens, but only in one plane.
The “optical axis” of a fixation target with a cylinder lens is an axis parallel to a direction of electromagnetic rays, which are parallel after passing through the cylinder lens.
The term “substantially parallel” describes electromagnetic rays with a parallel propagation direction. That means two electromagnetic rays are parallel if the propagation directions are identical. This is specifically the case for electromagnetic rays after passing through a cylinder lens if a source of the electromagnetic radiation in the focal plane is substantially parallel to the focal line of the cylinder lens, preferably arranged in the focal line of a cylinder lens. If sources of electromagnetic radiation are arranged in the focal line, the radiation is also perpendicular to the lens plane.

Two electromagnetic rays may also be substantially parallel if the propagation directions enclose an angle with each other, wherein this angle is less than approximately 10°, further preferably less than approximately 5°, particularly preferably less than approximately 2°, particularly preferably less than approximately 1°, particularly preferably less than approximately 0.1°, particularly preferably less than approximately 0.25, most preferably less than approximately 0.05°. If two electromagnetic rays pass the focal line in a cylinder lens and if the two electromagnetic rays are perpendicular to the focal line, they are substantially parallel after passing through the cylinder lens. If only one of the electromagnetic rays passes the focal line and the other ray does not pass the focal line or if both rays do not pass the focal line and if the two rays are not perpendicular to the focal line, the two rays are substantially parallel after passing through the cylinder lens if the respective distance from the focal line is less than a predetermined value. This can preferably be achieved in that a light source is not arranged in the focal line, but the light source is distanced from the focal line. Preferably, the distance of the light source from the focal line (or the focal plane) is less than approximately 5%, preferably less than approximately 2%, preferably less than approximately 1%, preferably less than approximately 0.5%, preferably less than approximately 0.1%, of the focal length of the cylinder lens. Advantageously, for the determination of the interpupillary distance, the apparatus allows a measurement accuracy of at least approximately ±0.2 mm, preferably of at least approximately ±0.05 mm, further preferably of at least approximately ±0.01 mm. For a Gullstrand's schematic eye (radius 12 mm), this corresponds to an angular displacement of less than approximately ±1°. This displacement is caused by a same deviation between the desired direction of the optical axis of the target and the actual direction thereof. Thus, for the above-mentioned distance of the light source from the focal line, preferably a deviation of the angular displacement of the eye of less than approximately 1° is made possible.
The terms “electromagnetic radiation” and “light” are used synonymously.
The term “substantially” can describe a slight deviation from a desired value, and in particular, a deviation within the framework of the manufacturing accuracy and/or within the framework of the necessary accuracy, so that an effect as present with the desired value is maintained. Therefore, the term “substantially” can include a deviation of less than approximately 30%, less than approximately 20%, less than approximately 10%, less than approximately 5%, less than approximately 2%, preferably less than approximately 1% from a desired values or desired position, etc. The term “substantially” comprises but is not limited to the term “identical”, i.e., without deviation from a desired value, a desired position, or the like.
The term “light field” describes electromagnetic radiation emitted from a flat object. The flat object can be part of a fixation target, for example. The flat object can be a curved surface of a cylinder lens, through which electromagnetic radiation exits from the cylinder lens. Although the electromagnetic radiation exits through the curved surface in this case, a test person looking at the light field perceives the light field as being emitted from a planar object. The light field can also be emitted from a surface of a diffuser, which is rectangular, for example. In other words, a “substantially rectangular light field” in its most general form describes a light field with a longitudinal extension and a width extension, wherein the longitudinal extension is greater than the width extension. It is also possible for the light field to be substantially square, i.e., the longitudinal direction is almost equal to the width extension. Consequently, the substantially rectangular light field may be the electromagnetic radiation emitted from a substantially rectangular surface, for example, an at least partially transparent surface illuminated from behind. In particular a substantially rectangular light field may be a light field whose projection onto a projection plane substantially is a rectangle, wherein the projection plane is perpendicular to the electromagnetic rays, which are parallel to each other, i.e. the projection plane is substantially perpendicular to the second plane (see below). The term “substantially rectangular” also includes a deviation from the rectangular shape, including but not limited to with rounded corners, substantially ellipse-shaped, preferably with a ratio of the long semiaxis to the short semiaxis of greater than 1:2. In the case of an elliptical target, in order to prevent the test person from departing from his habitual head and body posture to look at a target that is as long as possible, the target is preferably rectangular.
A “line” is not limited to a line in the mathematical sense. Instead, the term line also comprises a two-dimensional object with a finite length and a finite width. Thus, a line may be a rectangle with a small width compared to the length of the rectangle.
The term “homogenous light” and specifically along a direction describes that along this direction, light with a substantially equal light efficiency or luminous power is emitted by the illuminating device. At all points of the illuminating device along this direction, from which light is emitted, the emitted light has a substantially equal intensity. If the emitted light is substantially homogenous in this direction, the viewer cannot differentiate individual light sources, but sees a luminous line or a luminous stripe or luminous surface due to the finite extension of the illuminating device, which emits light of uniform intensity. This applies to a multitude of directions, in particular to a light-emitting surface.
The term “habitual head and body posture” provides the basis for an exact and tolerable spectacle lens centration. In particular, the “habitual head and body posture” substantially corresponds to a head and body posture of the test person, which is as natural as possible. For example, the test person can adopt the “habitual head and body posture” if he looks at himself in the mirror, as looking in the minor is an everyday and very common situation for every person. For example, the habitual head and body posture, compared to a view in the distance, can be achieved if the test person focuses his root of the nose in the minor image. The habitual head and body posture corresponds to the natural posture of the test person, which is determined by his physical and psychological state, habit, daily routine, work and leisure.

The test person has a relaxed neck posture and a healthy, substantially ideal head posture especially when the head is positioned exactly above the shoulders (and in the downward elongation exactly above the arch of foot). Thus the habitual head and body posture is preferably adopted while standing.

In a substantially ideal head posture, the head is substantially exactly above the shoulders (and in the downward elongation exactly above the arch of foot). The ears are perpendicular and are above the center of the shoulders. The neck is only slightly concave, i.e., bulged inwardly. In this position, the weight of the head is carried by the whole skeleton, via the spine. Since the neck muscles do not have to carry any weight, they are all soft and the head is freely movable on the spine. In all other head or neck postures, the neck muscles are chronically flexed, as they have to hold the weight of the head against gravity. Depending on whether the head is moved to the front or back or held inclined to the left or right, and whether the neck is bulged more strongly or less, different neck and body muscles are in a permanent contraction. This leads to different head and neck aches. At the same time, the neck has limited mobility, as the muscles have to fix the head in a specific posture and thus are available for movement only to a limited extent.

While sitting, according to different chairs/stools/other seats and due to various spine curvatures, there are different head and body postures depending on the sitting position. Classically, a differentiation is made between a centration according to the distant reference points and a centration according to the near reference points. Preferably, fitting takes place via the distant reference point or the centration cross, as the horizontal centration for near involves significantly greater uncertainties. In addition, high vertex powers result in a prismatic side effect that cannot be neglected any more. Thus, the near visual point drawn on the measurement lens or measurement spectacle lens shape does not coincide with the real visual point in the spectacle lens, since, in the finished spectacles, different accommodation and convergence requirements are placed on the spectacle wearer than while looking through the measurement lens or measurement spectacle lens shape (see Diepes as cited above). Therefore, centration is preferably performed according to the distant reference point, or the fitting point for progressive lenses is defined via the visual point at the zero direction of sight, i.e., when viewing in the distance, in the habitual head and body posture.

### DETAILED DESCRIPTION

An aspect of the preferred embodiments relates to a method of using at least one fixation target for aligning a direction of sight of the test person, in particular for aligning the pupils of the test person, wherein a flat extensive light field, and preferably a substantially rectangular light field, is generated by means of the fixation target, and the test person looks at the light field.

The fixation target can also be used for or when determining individual parameters of the test person. For example, the individual parameters of the test person include but are not limited to:
interpupillary distance;
monocular interpupillary distance;
corneal vertex distance according to reference point requirement and/or according to ocular center of rotation requirement;
monocular centration point distance;
centration point coordinates;
lens distance or spectacle lens shape distance;
decentration of the centration point;
vertical and horizontal lens size or vertical and horizontal spectacle lens shape size;
boxed center distance;
spectacle lens pantoscopic angle; and/or
fitting height.

Advantageously, the test person can be positioned in any arbitrary, predeterminable space direction or the test person's gaze can be aligned in any arbitrary, predeterminable space direction. Particularly advantageously, the visual behavior cannot be controlled by a person operating the apparatus.

In other words, the test person can focus on the light field at least partially. Thus, it is possible to align the test person's gaze on the basis of the light field, for measurement purposes, for example, such that the actual alignment of the pupils corresponds to a defined, predetermined visual behavior. Particularly advantageously, the direction of sight or the pupil position of the pupil(s) of the test person can be determined in the habitual head and body posture. Advantageously, the use of the light field allows the test person to assume his habitual head and body posture during fitting of a progressive lens, as in contrast to the use of a punctiform fixation target, such as a luminous point, the test person is only restricted slightly in his head posture, by the extension of the light field.

Thus, it is possible for the test person to look at the entire light field and to thereby assume his preferred, natural head posture. This is not possible if a fixation point in the form of a light point is used, as a light point restricts the direction of sight in all directions. Instead, the head posture is substantially predetermined by the fixation point in the form of a light point in this case, wherein a faulty positioning of the fixation point in the form of a light point inevitably causes a faulty alignment of the test person's visual behavior.

Similar to the use of a mirror image of the root of the nose as a fixation point, which also allows an alignment of the test person's gaze in his habitual head and body posture, that the method herein can prevent the visual behavior of the test person from being influenced by the person conducting the measurement. Also advantageously, a faulty influence of the person conducting the measurement can be reduced, which may occur if the person conducting the measurement determines the position of the fixation target. In contrast to the mirror image of the root of the nose, the apparatus disclosed herein offers greater freedom, in particular when adjusting the test person's direction of sight relative to the apparatus, preferably in the habitual head and body posture of the test person.

Further, the fixation target can preferably still be sufficiently recognized in the case of a visual defect of the test person, so that the test person can look at the light field of the fixation target. Optionally, the light field can appear to be wider than it is, wherein this can be neglected as long as the test person can look at the light field. This is often not possible if a fixation point is used. Particularly advantageously, the light field can be designed such that it is still sufficiently recognizable if the test person does not wear corrective spectacles. This can be achieved by a sufficient luminosity of the light field and/or color of the light of the light field.

Preferably, the test person can already be prepositioned. For example, a ground marking can be used to this end, which serves to position the test person at a predetermined position relative to the apparatus. The marking may be an adhesive label attached to the ground and/or a marking drawn onto the ground, e.g. in the form of a stripe and/or one or more crosses and/or of schematic feet, and the like. The marking may also be projected onto the ground by means of the apparatus. In particular, the marking is formed and arranged such that after positioning of the test person, at least one eye of the test person is already in the light field of at least one target, i.e., the test person can look at least one target with at least one eye. Consequently, the marking is matched to the extension of the light field of the fixation target.

In a preferred embodiment, the fixation target is formed such that the electromagnetic radiation of the light field is substantially diffused in a first predeterminable plane, and the electromagnetic radiation of the light field is substantially parallel in a second predeterminable plane, which is perpendicular to the first plane.

Further, the fixation target is preferably arranged and designed such that the test person can be positioned such that at least one pupil of the test person is substantially fully illuminated, i.e., that this pupil is substantially fully in the light field of the fixation target. This may also apply to the second pupil and optionally a further fixation target.

In other words, the ray path can be parallel in one direction and diffused in the direction perpendicular thereto. Thereby, the test person gets the impression of a luminous area. in the form of a luminous stripe, for example, and in particular of a luminous line in the direction of the diffused radiation. The extension of the light field can be greater than the stripe seen by the test person, but due to the substantially parallel radiation, the test person gets the visual impression of a stripe having substantially the width of the pupil of the test person. Preferably, the light field is significantly wider than the pupil of the test person, i.e., at least 2 times, 5 times, 10 times, 20 times as wide as the pupil of the test person. Thus, the test person can change his position without the visual impression changing, as long as the test person is in the light field of the fixation target and sees the light parallel in the second plane. In other words, the visible stripe “moves along” with the displacement of the test person.

Due to the formation of the light field, the direction of sight of the test person when viewing the light field is predetermined by the direction of the light field, if for example, by the direction of the parallel rays. If e.g. the first plane is a vertical plane in the frame of reference of the earth and the second plane is a horizontal plane in the frame of reference of the earth, the direction of sight of the test person is predetermined by the direction of the light of the light field in the horizontal direction. In the vertical direction, the direction of sight is limited by the vertical extension. Thus, the test person can assume his natural viewing posture within the light field.

In addition to the above, the test person will direct his view “to infinity” when looking at the light field of the fixation target due to the parallel electromagnetic rays. In other words, the test person perceives the light field as “infinitely” remote due to the parallel electromagnetic rays of the light field. Thus, the test person assumes a natural head and body posture corresponding to a natural vision in the distance, and specifically, straight ahead in the distance. Advantageously, the visual impression of the test person is substantially independent from the exact position of the eye in front of the fixation target, and in particular, in front of the light field as long as the test person looks at the parallel electromagnetic radiation. For example, the test person can displace his position in a direction parallel to the second plane, for example, in a horizontal direction, as long as they see the parallel electromagnetic radiation of the light field. In the vertical direction, the test person is free in his head movement due to the diffused electromagnetic radiation, i.e., the test person can move his head freely in the vertical direction if the first plane is a vertical plane, for example, and assume his natural head posture. Thus, the direction of sight is only predetermined in one space direction due to the direction of the parallel light, i.e., in the horizontal direction. If the light field is wide, the test person can slightly turn or displace his head, if necessary, wherein the visible stripe “moves along” in the horizontal displacement of the head. If the light field is narrow, the head posture of the test person is substantially limited to the narrow light field in the horizontal direction. In the exemplary vertical direction, the test person can select his direction of sight freely. This can be advantageous especially in the fitting of progressive lenses.

Advantageously, in contrast to the use of a punctiform fixation target, such as a luminous point, the head posture of the test person is only limited slightly, namely by the direction of the light field and by the extension of the light field in a direction in which the light field is preferably substantially homogenous.

Preferably, the test person can be positioned by means of the above-described marking such that the at least one eye is already in the light field of a target before the target is activated. Advantageously, this prevents the test person from changing his position (also the head posture) in order to bring his eyes in the region of the light field. The apparatus is preferably designed to take a turning of the head in the habitual direction of sight “straight” into account to compensate for it.

In other words, if a test person is asked to look at the light field, which may be formed in the form of a line or a stripe, his direction of sight in the plane in which the light field runs in a directed manner, i.e. in the second plane, adjusts in the direction of the light field while the gaze in the plane orthogonal thereto, i.e., in the first plane, remains unchanged. Advantageously, this may be used for controlling the visual behavior of the test person in particular for measurements of the individual parameters.

The above disclosure can apply to a multitude of first and a multitude of second planes. If, for example, the light field is substantially homogenous along a first direction, which lies in the first plane and is orthogonal to the second plane, the above disclosure applies to an infinite number of parallel second planes, namely for all parallel second planes intersecting the light field.

Preferably, the fixation target comprises a cylinder lens, and the first predeterminable plane is substantially parallel to a cylinder axis of the cylinder lens, and the second predeterminable plane is substantially perpendicular to the cylinder axis of the cylinder lens.

The cylinder axis is a longitudinal axis of the lens. The cylinder axis is parallel to the focal line of the cylinder lens.

Preferably, the cylinder axis is arranged in the frame of reference of the earth such that the cylinder axis is substantially parallel to a vertical plane.

In other words, the first plane is preferably substantially a vertical plane in the frame of reference of the earth. The second plane is preferably substantially a horizontal plane in the frame of reference of the earth.

Preferably, the light field is formed such that it is perceived as a stripe or line by the user.

Advantageously, a back surface of the cylinder lens can be substantially illuminated. In this case, the back surface is the surface facing towards a light source. If the light source is located in a focal line of the cylinder lens, the radiation propagating in a plane perpendicular to the focal line exits on a substantially parallel direction from a front surface of the cylinder lens. In the projection onto a projection plane, which is perpendicular to the propagation direction of the substantially parallel electromagnetic radiation, the resulting light field is a surface, in particular, a rectangle, which corresponds to the projection of the cylinder lens onto this projection plane. However, the test person perceives the light field merely as a stripe, since due to the parallel radiation direction of the light field in the second plane, the visual light field (in the second plane) is limited by the enlargement of the pupil of the test person. In the first plane, the radiation is diffused and therefore the visible light field (in the direction of the first plane) is limited by the extension of the cylinder lens, in particular dependent on the extension of the luminous surface and/or on the distance between the two elements. The projection plane is substantially parallel to the focal line and perpendicular to the propagation direction of the parallel radiation.

It is also possible for the back surface of the cylinder lens not to be illuminated completely. Instead, the illuminated region of the back surface of the cylinder lens can be vignetted by a diaphragm or the like. Advantageously, unfavorable effects, such as refraction, diffusion, etc., which may occur at the rim of the cylinder lens, or an image quality deteriorating toward the rim of the lens can be substantially avoided.

Preferably, the fixation target comprises an illuminating device, and the illuminating device generates electromagnetic radiation. Electromagnetic radiation is emitted at a multitude of points, and in particular, at an infinite number of points, along a first direction of the illuminating device if the illuminating device has a luminous surface, for example. Along the first direction, the intensity of the exiting electromagnetic radiation is substantially the same. Thus, the illuminating device has a homogenous luminous power or luminosity along the first direction, wherein the first direction is substantially perpendicular to the second plane.

Preferably the illuminating device comprises a luminous surface which generates a substantially homogenous, diffused light field, i.e., emits electromagnetic radiation of substantially homogenous intensity, and the luminous surface is arranged substantially perpendicular to the first plane and substantially perpendicular to the second plane. Thus, the intensity value of the electromagnetic radiation is substantially identical for all points.

In other words, the illuminating device comprises an extended light source or an extended light field formed on the basis of the cylinder lens. For example, the cylinder lens can have a flat surface as the backside and only have one curved surface. The luminous surface of the illuminating device is preferably substantially parallel to this flat surface and irradiates this flat surface with electromagnetic radiation.

In other words, the described light field can be generated by inserting a narrow, rectangular, diffused luminous surface into the focal plane of a cylinder lens such that the orientation of the diffused luminous surface is substantially parallel to the cylinder axis. Preferably, the focal line is arranged substantially in the middle of the luminous surface.

The “focal plane” of a cylinder lens is understood to be a plane that includes the focal line and is perpendicular to the optical axis.

The “focal line” of the cylinder lens is understood to be the line on which all focal points are located.

Preferably, the individual parameters of the test person are determined while the test person looks at the light field. In particular, the test person can focus on the light field at least one point.

Preferably, the fixation target is positioned such that the direction of the electromagnetic rays, which are substantially parallel to the second plane, is substantially perpendicular to a facial plane of the test person. The facial plane is understood to be the plane that includes the two pupils and is arranged vertically in the frame of reference of the earth.

Preferably, the light field has a length of at least approximately 40 mm along a first direction substantially perpendicular to the second plane.

In other words, along the vertical direction, the light field has a length of preferably between approximately 30 mm and approximately 70 mm, further preferably between approximately 35 mm and approximately 60 mm, particularly preferably at approximately 40 mm. In particular, it has been found that the light should not fall below a length of approximately 40 mm in the vertical direction.

Preferably, two fixation targets are used, wherein the two fixation targets are arranged and formed such that each eye of the test person perceives exactly one fixation target. Here, first the first eye can see a light field of a first fixation target and subsequently the second eye can see a light field of a second fixation target, wherein, for example, initially the first fixation target is operated and, after the first fixation target has been switched off, the second fixation target is operated. In other words, the two eyes can see or look at one fixation target each separately from each other. It is also possible to only operate one of the two fixation targets.

It is also possible for the two eyes to each see one fixation target at the same time, wherein the first eye sees the light field of the first fixation target and the second eye sees the light field of the second fixation target at the same time. The two light fields may be formed such that the test person sees the two light fields separately. For example, the light field of the first fixation target may have a different color than the light field of the second fixation target. The light field of the first fixation target may be red, the light field of the second fixation target may be green, or vice versa.

It is also possible for the test person to see the two light fields as one light field. The test person can then fuse the visual impressions of the two eyes. It is also possible to use a fixation target with two light fields.

Preferably, the fixation targets are arranged and formed such that the test person can fuse the respective images. In other words, the test person gets the visual impression of a common image of the two fixation targets.

Preferably, the illumination of the fixation targets can be controlled such that the test person only sees one fixation target each. In other words, two fixation targets can be mounted such that each eye of the test person perceives exactly one target. The test person can see the left fixation target or the right fixation target.

Here, the two fixation targets can be designed in color and/or brightness and/or direction of the light field, and further the line and/or parallelism of the optical axes of the fixation targets, and the like, that both eyes of the test person get the same visual impression and the test person can fuse the image.

Additionally or alternatively, this arrangement can be designed in a switchable manner, so that according to instructions by the person conducting the measurement, only one eye sees a light field without the test person having to change his position or direction of sight. Among others, this arrangement is particularly suitable for test persons with strabismus.

In an exemplary embodiment, a method is provided aligning a direction of sight of a test person, for determining the individual parameters of the test person. The method can include providing at least one light field in the form of at least a mentioned fixation target, and aligning a direction of sight of the test person on the basis of the light field by the test person looking at the light field.

Preferably, the method comprises the step of determining the individual parameters of the test person.

Another exemplary embodiment provides an apparatus for aligning the direction of sight of a test person, to determine individual parameters of a spectacle wearer. The apparatus can include at least one fixation target, wherein a flat extensive light field, in particular a substantially rectangular light field, can be generated by means of the fixation target, so that in the position of use of the apparatus, the light field can at least partially be seen by a test person.

Preferably, the fixation target is formed such that the electromagnetic radiation of the light field is substantially diffused in a first predeterminable plane, and the electromagnetic radiation of the light field in a second predeterminable plane, which is perpendicular to the first plane, is substantially parallel.

Preferably, the apparatus has two fixation targets and at least one image recording device, wherein the image recording device is preferably arranged between the two fixation targets. It is also possible for the apparatus to comprise two image recording devices arranged and used to create a stereo image of at least a subarea of the head of the test person, wherein the two image recording devices are preferably arranged such that a cyclopean eye of the two image recording devices is arranged between the fixation targets. The “cyclopean eye” describes the point or location from which an object appears to be viewed in a stereo image, wherein the stereo image is created by means of the image data of two cameras.

Preferably, the fixation target has a cylinder lens, wherein the cylinder axis is substantially parallel to the first plane and substantially perpendicular to the second plane. Preferably, the apparatus has an illuminating device, wherein the illuminating device comprises a substantially rectangular light-emitting surface. Preferably, the illuminating device comprises at least two light sources, in particular at least two LEDs. The illuminating device may also comprise any number of LEDs.

The at least two LEDs may be conventional LEDs. In particular, the at least two LEDs may be so-called homogenous LEDs. A homogenous LED is an LED that preferably produces a light field conveying a flat visual impression. In contrast to that, a conventional LED (which is not a homogenous LED) produces a light field conveying a substantially punctiform visual impression to a viewer, i.e., the test person. Preferably, the at least two homogenous LEDs are arranged such that they produce a substantially common light field, i.e., that the light fields of the first homogenous LED and the second homogenous LED (and optionally of the further homogenous LEDs) blend into one another and are free from a visible area, a visible stripe or a visible line between the individual light fields. Effectively, the test person only sees one light field. This applies to each fixation target analogously.

By analogy, each fixation target may comprise at least two cylinder lenses, wherein the above explanations concerning the at least two homogenous LEDs apply analogously.

Preferably, the illuminating device comprises at least one diffuser, wherein the light sources illuminate the diffuser such that the diffuser emits electromagnetic radiation with a substantially spatially, homogenously distributed intensity.

Preferably, the rectangular light-emitting surface of the illuminating device is at least partially arranged substantially in a focal plane of the cylinder lens. In particular, the light-emitting surface comprises the focal line of the cylinder lens. The light-emitting surface may be substantially parallel to the cylinder lens.

In other words, the luminous surface preferably coincides with the focal line so that the light being parallel perpendicularly to the cylinder axis is orthogonal to the plane of the lens.

Preferably, the fixation target, in particular the light field, is long enough in the direction of the cylinder axis that the exact position of the fixation target or the light field in this direction relative to the person to be measured does not have any substantial effect on his visual impression.

Preferably, the fixation target or the light field is wide enough in the lens plane in the direction perpendicular to the cylinder axis that the visual impression of the person to be measured is substantially independent both from the exact position of the fixation target or the light field and from his head posture. The lens plane is the plane that includes the optical center of the lens and is perpendicular to the optical axis of the lens.

Consequently, undesired influencing of the test person by external conditions and the adjustment of the apparatus by the person conducting the measurement can be advantageously reduced, and/or prevented. Preferably, the fixation targets are arranged such that the center distance (in the position of use of the fixation target substantially in the horizontal plane) of the two fixation targets corresponds substantially to the interpupillary distance of the test person. Preferably, the fixation targets are arranged such that the center distance corresponds to a conventional interpupillary distance, i.e., the center distance is approximately 64 mm, for example. The image recording device is preferably arranged between the two fixation targets, and the two fixation targets are preferably formed such that they have a smallest possible distance from the image recording device. In particular, the distance of each fixation target from the image recording device is preferably less than approximately 7 mm, preferably less than approximately 5 mm, preferably less than approximately 3 mm, preferably less than approximately 1 mm, preferably equal to approximately 0 mm.

The rectangular light-emitting surface can be a diffuser, for example, in particular a diffuser illuminated from behind.

As the width of the rectangular surface or the diffuser determines the angular distribution in the direction of the parallel light, i.e., the direction of the electromagnetic radiation in the second plane, the width of the rectangular surface of the diffuser can preferably be adjusted to the desired accuracy. Moreover, the angular distribution is influenced by the actual distance of the luminous surface from the focal plane. The tolerance for the position of this light source, in particular, the luminous surface in the direction of the optical axis of the cylinder lens (i.e. in particular the distance of the rectangular surface or the diffuser from an adjacent surface of the cylinder lens) can also be selected correspondingly on the basis of the desired angle accuracy of the light exiting the fixation target, i.e., the light of the light field.

The exit angle of the parallel course to the lens plane is determined by the distance of the arranged, diffused luminous surface from the focal line. Accordingly, the required lateral positioning accuracy of the luminous surface in the focal plane can be adapted to the desired angular accuracy.

The luminous surface can be realized by LEDs, other illuminants and/or a diffuser plate illuminated from behind. For delimiting the width of the luminous line, a slit-shaped diaphragm (also in the focal plane) with a defined width can be employed.

In order to avoid an influencing of the direction of sight of the test person in the direction of the cylinder axis according to the disclosure herein, the light field in the direction of the cylinder axis is not only diffused, but also sufficiently homogenous. The luminous surface is designed accordingly homogenously.

Preferably, the image recording device, and in particular, a center of an aperture of the image recording device, is distanced from the at least one fixation target by between approximately 5 mm and approximately 40 mm, and specifically equal to approximately 17 mm, for example.

Preferably, the fixation target is arranged such that the cylinder axis is arranged substantially vertically in the frame of reference of the earth. Advantageously, the test person is thus substantially not influenced in his vertical view and eye alignment, i.e. the test person can assume his natural head and/or body posture in the vertical direction.

Moreover, the fixation target can be arranged such that the optical axis of the fixation target is orthogonal to the facial plane of the test person, so that he looks “straight ahead”.

Thus, it can be advantageously achieved that the test person automatically assumes the so-called habitual head and/or body posture, i.e., that his alignment of body and/or head and/or pupils corresponds to the alignment(s) the test person assumes casually when looking straight ahead to infinity without being influenced.

Preferably, the apparatus has at least one presenting means for presenting at least one characteristic point of a spectacle lens, wherein the at least one image recording device is designed and arranged to generate image data of the at least one presenting means and at least of subareas of a spectacle lens and a spectacle frame of the test person, and wherein the apparatus further comprises a data processing device designed to determine a position of a spectacle lens relative to the spectacle frame on the basis of the image data.

Preferably, the apparatus comprises at least two image recording devices designed and arranged to each generate image data at least of subareas of the head of the test person; a data processing device with a user data determining device designed to determine user data of at least a subarea of the head or at least a subarea of a system of the head and spectacles, arranged thereon, in the position of wear of the test person on the basis of the generated image data, wherein the user data comprise location information in the three-dimensional space of predetermined points of the subarea of the head or the subarea of the system, and a parameter determining device designed to determine at least part of the optical parameters of the test person on the basis of the user data; and a data output device designed to output at least part of the determined optical parameters of the test person.

User data may in particular comprise data of the test person, such as location information for at least one of the following points:
intersection points of a horizontal plane in the frame of reference of the user with the spectacle frame rims of the spectacles, wherein the horizontal plane of the user intersects both pupils of the user and is parallel to a predetermined zero line of sight of the user;
intersection points of a vertical plane in the frame of reference of the user with the spectacle lens rims and/or the spectacle frame rims of the spectacles, wherein the vertical plane of the user is perpendicular to the horizontal plane of the user and parallel to the predetermined zero line of sight of the user and intersects a pupil of the user;
at least one pupil center point;
delimitations of at least one spectacle lens of the user according to dimensioning in the boxing system;
spectacle center point of the spectacle frame of the spectacles.

The optical parameters are in particular the individual parameters of the test person.

Preferably, the apparatus comprises at least two image recording devices, each designed and arranged to generate comparative image data of at least a subarea of the head of the test person in the absence of the spectacles and/or in the absence of the at least one spectacle lens and of at least a subarea of an auxiliary structure, and generate image data of a substantially identical subarea of the head of the test person with spectacles arranged thereon and/or at least one spectacle lens arranged thereon and of at least the subarea of the auxiliary structure; a data processing device designed to determine the position of the spectacles and/or of the at least one spectacle lens relative to the pupil center point of the corresponding eye of the test person in the zero direction of sight on the basis of the image data, on the basis of the comparative image data and on the basis of at least the subarea of the auxiliary structure, and a data output device designed to output the position of the spectacles and/or of the at least one spectacle lens relative to the pupil center point of the corresponding eye of the test person in the zero direction of sight.

Preferably, the fixation target can be arranged in the apparatus such that the optical axis of the fixation target is preferably parallel to an optical axis or effective optical axis of one or more image recording devices.

If two or more image recording devices are present, by means of which the three-dimensional data, i.e. stereo images, are created, the optical axis of the fixation target can preferably be aligned in parallel with an optical axis of a cyclopean eye of these two or more image recording devices.

Preferably, one of the image recording devices is arranged between two fixation targets.

The disclosure herein is not limited to the above-described aspects and embodiments. Instead, individual features of the aspects and/or embodiments can be combined separately with each other in an arbitrary manner and in particular thus form new embodiments of the different aspects. In other words, the above explanations regarding the individual features of the apparatus analogously also apply to the use and/or the method, and vice versa.

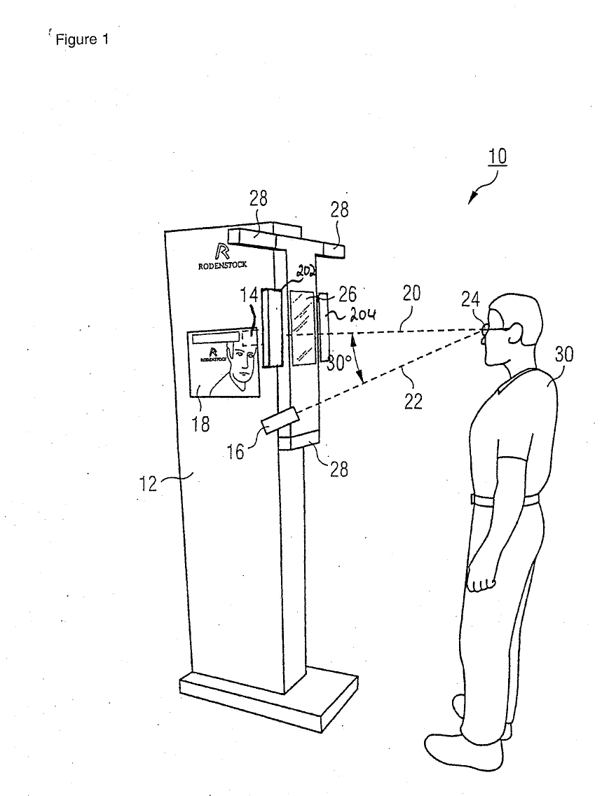

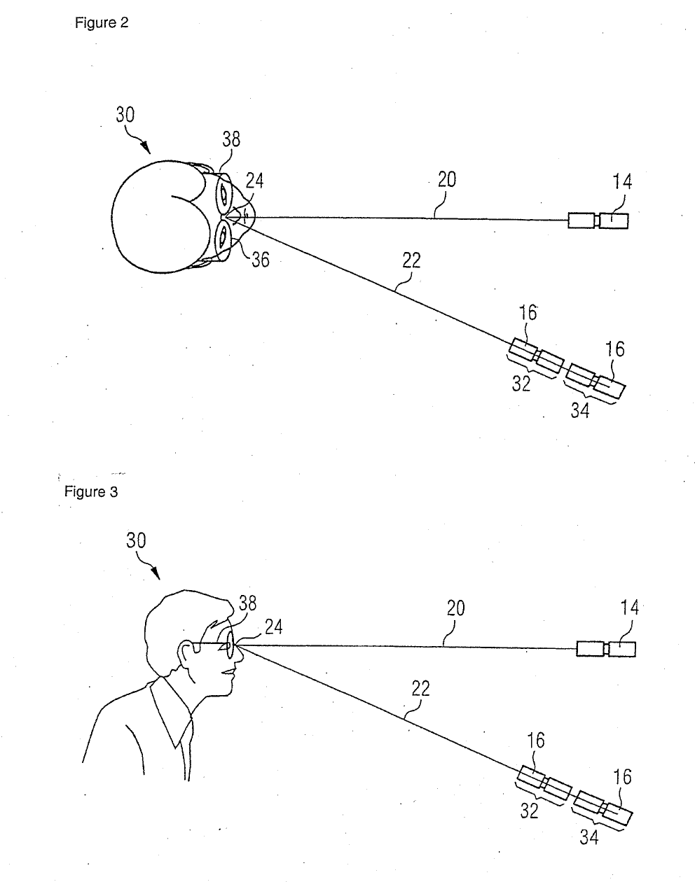

FIG. 1 shows a schematic perspective view of an apparatus 10 according to a preferred embodiment of the preferred embodiments. The apparatus 10 comprises an arrangement device in the form of a housing or a column 12, on which a first image recording device in the form of an upper camera 14 and a second image recording device in the form of a lateral camera 16 are arranged. Moreover, a data output device in the form of a monitor 18 is integrated in the column 12. The upper camera 14 is preferably located in the interior of the column 12, for example as shown in FIG. 1, at least partially at the same height as the monitor 18. In the operating position, the upper camera 14 and the lateral camera 16 are arranged such that an effective optical axis 20 of the upper camera 14 intersects with an effective optical axis 22 of the lateral camera 16 in an intersection point 24. The intersection point 24 of the effective optical axes 20, 22 preferably is the point of the root of the nose (compare FIG. 2) or the center of the bridge (not shown).

The upper camera 14 is preferably arranged centrally behind a partially transparent minor 26. The image data of the upper camera 14 are generated through the partially transparent minor 26. The image data (referred to as images in the following) of the upper camera 14 and the lateral camera 16 are preferably output on the monitor 18. Furthermore, three illuminants 28 are arranged on the column 12 of the apparatus 10. The illuminants 28 can be fluorescent rods, such as fluorescent tubes, for example. However, the illuminants 28 may also include one or more incandescent lamps, halogen lights, light-emitting diodes, etc.

In the preferred embodiment of the apparatus 10 illustrated in FIG. 1, the effective optical axis 20 of the upper camera 14 is arranged in parallel to the zero direction of sight of a user 30. The zero direction of sight corresponds to the visual axis of the eyes of the user in the primary position. The lateral camera 16 is arranged such that the effective optical axis 22 of the lateral camera 16 intersects the effective optical axis 20 of the upper camera 14 in an intersection point 24 at an intersection angle of approximately 30°. The intersection point 24 of the effective optical axes 20, 22 preferably is the point of the root of the nose (compare FIG. 2) of the user 30. In the preferred embodiment of the apparatus 10, this means that the effective optical axis 22 also intersects the zero direction of sight at an angle of 30°. The intersection angle of 30° is a preferred intersection angle. Other intersection angles are also possible. However, the intersection angle is preferably less than approximately 60°.

Furthermore, it is not necessary for the effective optical axes 20, 22 to intersect. Instead, it is also possible that the minimum distance of the effective optical axes from the location of the root of the nose of the user 30 is less than approximately 10 cm, for example. Furthermore, it is possible that a further lateral camera (not shown) is arranged on the column 12, wherein the further lateral camera lies diagonally opposite to the lateral camera 16, for example.

In a further preferred embodiment, the upper camera 14 and the lateral camera 16 may be arranged such that their positions and in particular their effective optical axes can be tailored to the body size of the user 30, for example. The determination of the relative positions of the cameras 14, 16 to each other can be performed by means of a known calibration method.

Moreover, the cameras 14, 16 may be designed, for example, to generate single images of a subarea of the head of the user 30. However, it is also possible to record video sequences by means of the cameras 14, 16 and to use these video sequences for further analysis. Preferably, however, single images are generated by the cameras 14, 16 and the single images are used for the further analysis, the upper camera 14 and the lateral cameras 16 being time synchronized, i.e. they record or generate images of the preferably identical subarea of the head of the user 30 is a synchronized manner. Furthermore, it is possible that images of different areas of the head of the user 30 are recorded by both cameras 14, 16. The images of the two cameras contain at least one identical subarea of the head of the user 30 though.

In the operating position, the user is preferably situated or positioned such that his view is directed toward the partially transparent 26 mirror, wherein the user looks at the image of the root of this nose (compare FIG. 2) in the minor image of the partially transparent mirror 26.

The column 12 may have an arbitrary other shape or present a different type of housing in which that cameras 14, 16 and e.g. the illuminants 28, the partially transparent minor 26, and the monitor 18 are arranged.

In the operating position, the distance between the partially transparent minor 26 and the user 30 is only between approximately 50 and 75 cm, wherein the user 30 stands in front of the minor or is seated in front of the partially transparent minor 26 in accordance with an activity in which the user 30 wears spectacles, for example. Thus, the employment of the preferred apparatus is also possible in restricted spatial conditions. Accordingly, the apparatus 10 may be designed such that the positions of the upper camera 14 and the lateral 16 and e.g. also of the partially transparent mirror 26 and the illuminants are arranged to be adjustable in height. The upper camera 14 may therefore also be arranged above or below the mirror 18. Moreover, it is also possible to tilt or rotate the column 12 and/or the upper camera 14, lower camera 16, partially transparent mirror 26, and illuminants 28 arranged on the column 12, about a horizontal axis in the frame of reference of the earth.

According to a further preferred embodiment, for example the lateral camera 16 may be replaced by a pattern projection device, such as a conventional projector, and the three-dimensional user data may be determined by means of a conventional method, such as the phase-measuring triangulation.

FIG. 2 shows a schematic plan view of preferred arrangements of the cameras 14, 16 in the operating position and the positioning of a user 30 in the operating position. As shown in FIG. 2, projections of the effective optical axes 20, 22 intersect on a horizontal plane in the frame of reference of the earth at an angle of 23.5°. The intersection angle between the effective optical axes 20, 22 in the plane which is spanned by the two effective optical axes 20, 22 is 30°, as shown in FIG. 1. The intersection point 24 of the effective optical axes 20, 22 corresponds to the location of the root of the nose of the user 30. As can also be seen from FIG. 2, a position of the lateral camera 16 can be changeable along the effective optical axis 22, for example. The position 32 of the lateral camera 16 e.g. corresponds to the position as shown in FIG. 1. The lateral camera 16 may also be arranged in an offset manner along the effective optical axis 22 at a position 34, preferably the lateral camera 16 can be positioned in an arbitrary manner. However, at least one pupil (not shown) of the user as well as at least one spectacle lens rim 36 or a spectacle frame rim 36 of spectacles 38 of the user have to be imaged in the image data generated by the lateral camera 16. Furthermore, the pupil has to be imaged preferably completely within the spectacle frame or lens rim 36 of the spectacles 38. Analogously, the upper camera 14 can be positioned differently as well.

Furthermore, if merely the position of one or two spectacle lenses relative to the spectacle frame is to be determined and checked, for example, it is not necessary for the user 30 to wear the spectacles 38 on his head for determining the position of the spectacle lens relative to the spectacle frame. Instead, the position of the spectacle lens relative to the spectacle frame can also be determined independent from the user 30. For example, the spectacles 38 may be placed on a tray, such as a table (not shown). Consequently, the apparatus can thus be designed differently as well, e.g. have different dimensions. In particular, the apparatus can be smaller than illustrated in FIG. 1. For example, the apparatus may merely have the two cameras 14, 16, which may be arranged substantially stationary with respect to each other. The cameras are designed to be connectable to a computer, so that a data exchange is possible between the cameras 14, 16 and the computer. For example, the apparatus may also be designed in a mobile manner. In other words, the image recording devices, i.e. the cameras 14, 16, may be arranged separately from the data processing device, i.e. the computer, in particular be accommodated in separate housings.

It is also possible for the spectacles to be worn by a person other than the actual user.

FIG. 3 shows a schematic sectional side view of the arrangement of the cameras 14, 16 in the operating position as well as a position of the user 30 in the operating position, as shown in FIG. 1. As already shown in FIG. 2, the lateral camera 16 may be positioned along the effective optical axis, for example, at the position 32 or at the position 34. Furthermore, FIG. 3 shows the projection of the effective optical axes 20, 22 onto a vertical plane in the frame of reference of the earth. The angle between the effective optical axes 20, 22 is e.g. 23.5°, which corresponds to an intersection of 30° in the plane spanned by the effective optical axes 20, 22.

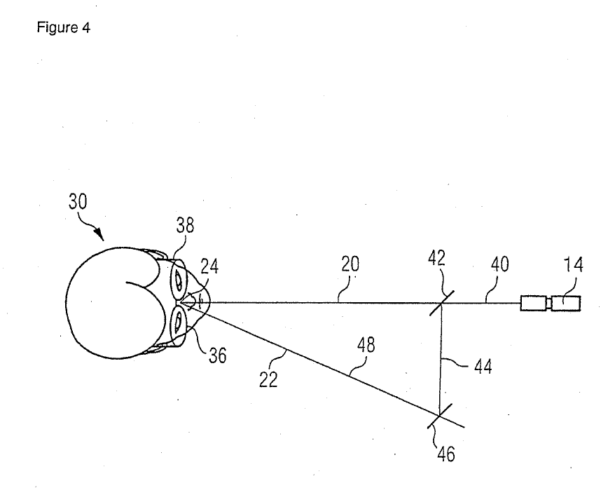

FIG. 4 shows a sectional plan view of a second preferred embodiment of the apparatus 10. Instead of two cameras, only the upper camera 14 is used. The upper camera 14 has an optical axis 40. The optical axis 40 corresponds to a line that extends from a center point of the aperture (not shown) of the upper camera 14 and is perpendicular to the plane of the aperture (not shown) of the upper camera 14.

Starting from the upper camera 14, a beam splitter 42 is located in the beam path of the camera 14 in the direction of the optical axis 40. The beam splitter 42 is for example designed such that it may change between two modes of operation:
the beam splitter 42 is either almost completely reflective, or
the beam splitter is almost completely transparent to light.

For example, if the beam splitter 42 is completely transparent to light, the optical axis 40 of the upper camera 14 is not deflected, but intersects the head of the user 30 at an intersection point 24. In this case, the effective optical axis 20 corresponds to the optical axis 40 of the upper camera 14. However, if the beam splitter 42 is completely reflective, the optical axis 40 of the upper camera 14 is deflected by the beam splitter 42 according to known optical laws, as show in FIG. 4. For example, the optical axis 40 is deflected at an angle of 90° into a first deflected subregion 44 of the optical axis 40 of the upper camera 14. The first deflected subregion 44 intersects a further optical element, for example a deflection minor 46. Thereby, the first deflected subregion 44 of the optical axis 40 is again deflected into a second deflected subarea 48 of the optical axis 40 according to the conventional optical laws. The second deflected subarea 48 of the optical axis 40 intersects the head of the user 30. The second deflected subarea 48 of the optical axis 40 corresponds to the effective axis 22 of the upper camera 14, for the case in which the beam splitter 42 is completely reflective.

Images of the subarea of the head of the user 30 are generated by the upper camera 14 in a time-shifted manner, wherein the images are either generated with a completely reflective beam splitter 42 or with a completely transparent beam splitter 42. In other words, two images of the subarea of the head of the user 30 can be generated by means of the upper camera 14, said images corresponding to the images as can be generated according to FIG. 1, 2, or 3. However, the images in this preferred embodiment are generated in a time-shifted manner by one image recording device, the upper camera 14.

FIG. 5 shows a schematic view of image data as are generated by the upper camera 14, i.e. a schematic front view of a subarea of the head of the user 30, wherein only two spectacle lenses 50 as well as a spectacle frame 52 as well as a right eye 54 and a left eye 56 of the user 30 are illustrated. A pupil center point 58 of the right eye 54 and a pupil center point 60 of the left eye 56 are shown as user data in FIG. 5. Furthermore, FIG. 5 shows a delimitation 62 of the spectacle frame 52 for the right eye 54 and a delimitation 64 of the spectacle frame 52 for the left eye 56 in the boxing system, as well as intersection points 66 of a horizontal plane in the frame of reference of the user with the spectacle frame rim 52 in respect to the right eye 54 as well as intersection points 68 of a vertical plane in the frame of reference of the user 30 perpendicular to the horizontal plane of the user 30. The horizontal plane is illustrated by the dashed line 70, the vertical plane by the dashed line 72.

Analogously, intersection points 74 of a horizontal plane and intersection points 76 of a vertical plane for the left eye 56 are shown in FIG. 5, wherein the horizontal plane is illustrated by the dashed line 78 and the vertical plane by the dashed line 80.

Preferably, the pupil center points 58, 60 are determined automatically by a user data positioning device (not shown). To this end, reflexes 82 are used, which arise on the corneas of the respective eyes 54, 56 due to the illuminants 28. Since according to the embodiment of the apparatus 10 preferred embodiments shown in FIG. 1, three illuminants 28 are arranged, for example, three reflexes 82 are imaged per eye 54, 56. The reflexes 82 arise for each eye 54, 56 directly at the penetration point of a respective illuminant visual axis on the cornea. The illuminant visual axis (not shown) is the straight connecting line between the location of the respective illuminant 28, which is centrally imaged on the retina, and the respective pupil center point 58, 60 of the corresponding eye 54, 56. The elongation of the illuminant visual axis (not shown) passes through the optical ocular center of rotation (not shown). Preferably, the illuminants 28 are arranged such that they lie on a conical cylindrical surface, the apex of the cone being located at the pupil center points 58 and 60 of the right eye 54 and the left eye 56, respectively. Starting from the cone apex, the axis of symmetry of the cone is arranged in parallel to the effective optical axis 20 of the upper camera 14, wherein the three illuminants 28 are further arranged such that straight connecting lines of the cone apex and the respective illuminant 28 merely intersect in the cone apex.

The pupil center points 58 and 60 of the right eye 54 and the left eye 56, respectively, can be determined on the basis of the reflexes 82 for the right eye 54 and the left eye 56.

FIG. 5a shows a schematic view of image data, similar to FIG. 5, as are generated by the upper camera 14, i.e. a schematic front view of a subarea of the spectacles 38, wherein two spectacle lenses 154, 156 and one spectacle frame 52 are illustrated. FIG. 5a shows a delimitation 62 of the spectacle frame 52 for the right eye 154 and a delimitation 64 of the spectacle frame 52 for the left eye 156 in the boxing system, as well as intersection points 66 of a horizontal plane in the frame of reference of the earth with the spectacle frame rim 52 in respect to the right spectacle lens 154 as well as intersection points 68 of a vertical plane in the frame of reference of the earth perpendicular to the horizontal plane. The horizontal plane is illustrated by the dashed line 70, the vertical plane by the dashed line 72.

Analogously, intersection points 74 of a horizontal plane and intersection points 76 of a vertical plane for the left spectacle lens 156 are shown in FIG. 5, wherein the horizontal plane is illustrated by the dashed line 78 and the vertical plane by the dashed line 80.

Preferably, the presenting means are automatically determined by the data processing device (not shown) in the form of adhesive labels 150.

Moreover, two presenting means 150 are exemplarily shown in FIG. 5a. The presenting means 150 may be a so-called saddle point, which is formed as an adhesive label 150, for example. The presenting means 150 may also be a single-color point 150, which can be arranged on the spectacle lens (shown in FIG. 6a) either as an adhesive label or which is drawn directly onto the spectacle lens (shown in FIG. 6a) e.g. with a pencil.

FIG. 5b is an illustration similar to FIGS. 5 and 5a, wherein one saddle point 53 as a preferred auxiliary point and two saddle points 153, 253 as preferred presenting means are illustrated in addition.

Each saddle point 53, 153, 253 may be an adhesive label, for example. It is also possible to use two saddle points 53, wherein one saddle point is associated with the left eye (not shown), and one saddle point with the right eye (not shown).

Particularly preferably, 9 saddle points 53, 153, 253 (not shown) are used, wherein three saddle points 153 are arranged on the one spectacle lens (not shown), three saddle points 253 are arranged on the other spectacle lens (not shown), and three saddle points 53 are arranged on the head, for example the forehead, of the user (not shown), in order to determine a position of each spectacle lens relative to the corresponding eye, i.e. the corresponding pupil or the corresponding pupil center in the three-dimensional space.

Preferably, the saddle point 53 is automatically recognized and determined by a user data positioning device (not shown).

FIG. 6 shows a schematic view of the image data of the lateral camera 16 according to FIG. 5. Since the lateral camera 16 is located laterally below the subarea of the head of the user 30, intersection points of a horizontal and a vertical plane with the rims of the spectacle frame 52 do not lie on horizontal or vertical straight lines, as is the case in FIG. 5. Instead, straight lines, on which intersection points with the horizontal plane and the vertical plane lie, are projected onto inclined lines 84 due to the perspective view of the lateral camera 16. Therefore, the horizontal plane 70 and the vertical plane 72 intersect the rim 36 of the spectacle frame 52 at the locations where the projected straight lines 84 each intersect the rim 36 of the spectacle frame 52. Analogously, the pupil center points 58, 60 may also be determined by means of the reflexes 83 on the basis of the image data illustrated in FIG. 6.

By means of the intersection points 66, 68, 74, 76 shown in FIGS. 5 and 6 and the pupil center points 58, 60, three-dimensional coordinates of the system of spectacles 30 and eye(s) 54, 56 can be generated. Moreover, specific points in the boxing system may be used to determine the three-dimensional coordinates. Alternatively, the three-dimensional coordinates may also be at least partially generated using the points determined according to the boxing system if necessary. On the basis of the positions in the image data, i.e. the intersection points 66, 68, 74, 76 and the pupil center points 58, 60, knowing the positions of the upper camera 14 and the lateral camera 16, location relations may be generated in the three-dimensional space in the system of eye(s) 54, 56 and spectacles 30. The intersection points 66, 68, 74, 76 or the pupil center points 58, 60 can be determined by an optician and input by means of a computer mouse (not shown). Alternatively, the monitor 18 may be designed as a “touch screen”, and the intersection points 66, 68, 74, 76 or the pupil center points 58, 60 can be determined and input directly by means of the monitor 18. Alternatively, these data can also be generated automatically by means of image recognition software. In particular, it is possible to perform a software-supported image analysis with subpixel precision. According to a further embodiment, the positions of further points of the spectacles 38 can be determined and used to determine the optical parameters in the three-dimensional space.

Optical parameters of the user 30 can be determined on the basis of the three-dimensional user data of the system of eyes 54, 56 and spectacles 30, wherein head and eye movements can be taken into account in this determination. To this end, for example, a multitude of images is generated, wherein the user 30 performs a head movement or tracks a moving object with his eyes. Alternatively, it is also possible to generate images during discrete head or eye excursions, which may be used e.g. for determining a convergence behavior of the eyes or for determining differences in the eye excursion behavior. As shown in FIG. 1, the user is preferably positioned in a primary position and, as can be taken from FIG. 2, for example the effective optical axis 20 of the upper camera 14 and the center parallel lines of the visual axes of the eyes 54, 56 in the primary position are identical. A further embodiment of the apparatus 10 is designed such that merely one eye, i.e. either the right eye 54 or the left eye 56, is imaged both by the upper camera 14 and the lateral camera 16. The optical parameters of the user 30 are determined on the basis of said one eye 54, 56, and the optical parameters for both eyes 54, 56 are determined assuming symmetry.

Advantageously, according to the apparatus 10, the optical parameters, i.e. for example interpupillary distance, corneal vertex distance, face form angle, pantoscopic angle, and fitting height, can be determined for a user 30 whose exe excursion does not correspond to the zero direction of sight. Instead, the user 30 looks at the image of the bridge of his nose in the partially transparent mirror 26 at a distance of approximately 50 to 75 cm according to the preferred embodiments. In other words, the user 30 is located at a distance of approximately 50 to approximately 75 cm in front of the partially transparent minor 26, and looks at the image of his face in the partially transparent mirror 26, in particular at the root of his nose. The position of the eyes 54, 56 resulting from the object looked at, i.e. the convergence of the eyes 54, 56, may be taken into account in the determination of the optical parameters, and rotations of the eyes can e.g. be compensated for when determining the optical parameters, wherein for example a virtual zero direction of sight can be determined considering the actual eye excursion, and the optical parameters of the user can be determined on the basis of the virtual zero direction of sight, i.e. the determined and unmeasured zero direction of sight. Advantageously, the distance between the user 30 and the cameras 14, 16 can thus be small. In particular, it is also possible to approximately predetermine the optical parameters. Furthermore, the spectacles 38 may be prefitted and the optical parameters may be determined using the apparatus 10 for the prefitted.

Moreover, according to a further preferred embodiment, the apparatus 10 is designed to calculate the pantoscopic angle of the spectacles 38 for each eye 54, 56 from the angle between the straight line through the upper intersection point 68 and the lower intersection point 68 of the vertical intersection plane 72 with the rim 36 of the spectacle frame 52 in the three-dimensional space. In addition, a mean pantoscopic angle can be determined from the pantoscopic angle determined for the right eye 54 and the pantoscopic angle determined for the left eye 56. Furthermore, a warning notification may be output if the pantoscopic angle of the right eye 54 deviates from the pantoscopic angle of the left eye 56 by at least a predetermined maximum value. Such a notification may be output by means of the monitor 18, for example. Analogously, the face form angle and the corneal vertex distance or the interpupillary distance may be determined from the three-dimensional data set for the right eye 54 and the left eye 56 as well as mean values thereof, and notifications may optionally be output via the monitor 18 if the deviations of the values for the right eye 54 and the left eye 56 each exceed a maximum value.

The corneal vertex distance can selectively be determined according to reference point requirement or according to ocular center of rotation requirement. According to the reference point requirement, the corneal vertex distance corresponds to the distance of the vertex of the spectacle lens 50 from the cornea at the penetration point of the visual axis of the eye in the zero direction of sight. According to the ocular center of rotation requirement, the corneal vertex distance corresponds to the minimum distance of the cornea from the spectacle lens 50.

Furthermore, the apparatus 10 can be designed such that the fitting height of the spectacle lens 50 is calculated on the basis of a distance of the penetration point of the visual axis of an eye 54, 56 in the primary position with a lens plane of a spectacle lens 50 from a lower horizontal tangent in the lens plane. A lower horizontal tangent is e.g. the line 84 of the delimitation 62, 64 according to the boxing system. Preferably, the apparatus 10 is designed such that a three-dimensional closed polyline is determined for the lens shape of the spectacle lens 50 from points on the rim 36 of the spectacle frame 52 for each eye 54, 56, wherein an averaged polyline for the lens shape can be determined from polylines of the respective spectacle lenses 50 of the right eye 54 and the left eye 56.

Alternatively, it is also possible that instead of averaging the values of the optical parameters, which are determined for the right eye 54 and the left eye 56, the optical parameters or the polylines for the lens shape are merely determined for the spectacle lens 50 of one of the eyes 54, 56, and these values are also used for the other of the eyes 54, 56.

Furthermore, the apparatus according to a preferred embodiment can be used to generate images of the user 30 and to superimpose image data of a multitude of frame and/or spectacle lens data on these images, whereby it is possible to advise the user 30 optimally. In particular, materials, layers, thickness, and colors of the spectacle lenses, the image data of which are superimposed on the generated image data, can be varied. Therefore, the apparatus 10 can be designed to provide fitting recommendations, in particular optimized individual parameters, for a multitude of different spectacle frames or spectacle lenses.

FIG. 6a shows a schematic view of the image data of the lateral camera 16 according to FIG. 5a, similar to the illustration according to FIG. 6. As the lateral camera 16 is located laterally below the subarea of the head of the user 30, the intersection points of a horizontal and a vertical plane with the rims of the spectacle frame 52 do not lie on horizontal and vertical straight lines, respectively, as this is the case in FIG. 5a. Instead, straight lines, on which intersection points with the horizontal plane and the vertical plane lie, are projected onto inclined straight lines 84 due to the perspective view of the lateral camera 16. Therefore, the horizontal plane 70 and the vertical plane 72 intersect the rim 36 of the spectacle frame 52 at the locations where the projected straight lines 84 each intersect the rim 36 of the spectacle frame 52.

By means of the intersection points 66, 68, 74, 76 shown in FIGS. 5 and 6, three-dimensional coordinates of the spectacles 30 can be generated. Moreover, the box dimension in the three-dimensional space can be determined on the basis of the three-dimensional coordinates.

As an alternative to the generation of data or coordinates in the three-dimensional space on the basis of image data recorded under different directions, the image data may also be recorded under only one direction, and the three-dimensional data may be generated on the basis of additional data. For example, it may be sufficient to record image data substantially from the front and to additionally indicate the face form angle and/or the pantoscopic angle of the spectacles and/or the corneal vertex distance and/or the head rotation, etc. On the basis of the image data and the additional data, the position in the three-dimensional space, in particular of the spectacle lens in front of the eye, can be determined.

The intersection points 66, 68, 74, 76 or the saddle point 150 can be determined by an optician and input by means of a computer mouse (not shown). Alternatively, the monitor 18 may be designed as a “touch screen”, and the intersection points 66, 68, 74, 76 or the saddle point 150 can be determined and input directly by means of the monitor 18. Alternatively, these data can also be generated automatically by means of image recognition software. In particular, it is possible to perform a software-supported image analysis with subpixel precision. According to a further embodiment, the positions of further points of the spectacles 38 can be determined and used to determine the optical parameters in the three-dimensional space.

FIGS. 5a and 6a merely show two saddle points 150. Preferably, four saddle points, particularly preferably six saddle points (not shown) are arranged, wherein two or three saddle points are arranged on each spectacle lens in order to enable an unambiguous determination of the position of each spectacle lens in the three-dimensional space.

The box dimension of the spectacles 30 in the three-dimensional space can be determined on the basis of the three-dimensional user data of the spectacles 30, and in particular the position of the saddle point 150 in the boxing system (in the three-dimensional space).

Furthermore, a lower tangent 86 is drawn to the spectacle frame 52 in FIG. 5a and FIG. 6a. The lower tangent 86 is a part of the delimitation 62, 64 of the boxing system.

The spectacles may also be designed such that pupils (not shown) are imaged.

A further embodiment of the apparatus 10 is designed such that merely a side, i.e. either the right side corresponding to the right eye or the left side corresponding to the left eye, is imaged both by the upper camera 14 and the lateral camera 16. The optical parameters of the user 30 are determined on the basis of said one side, and the optical parameters for both sides are determined assuming symmetry.

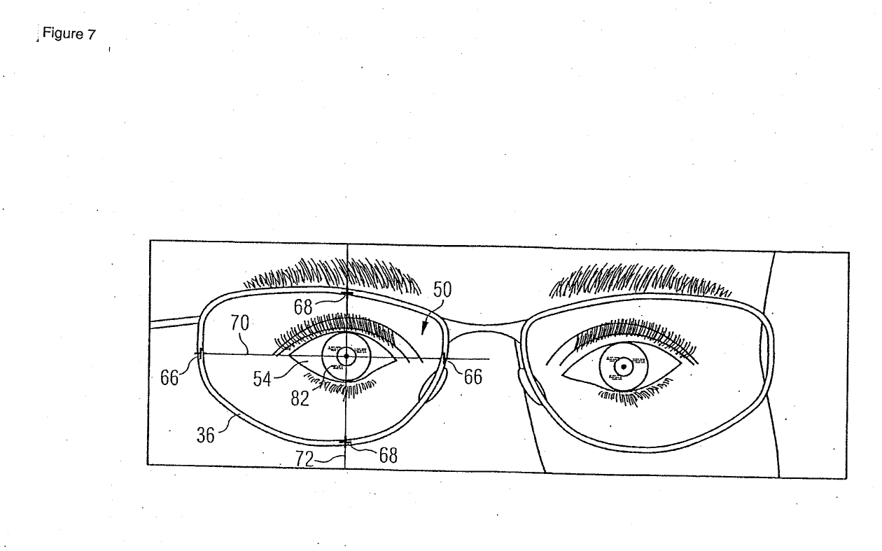

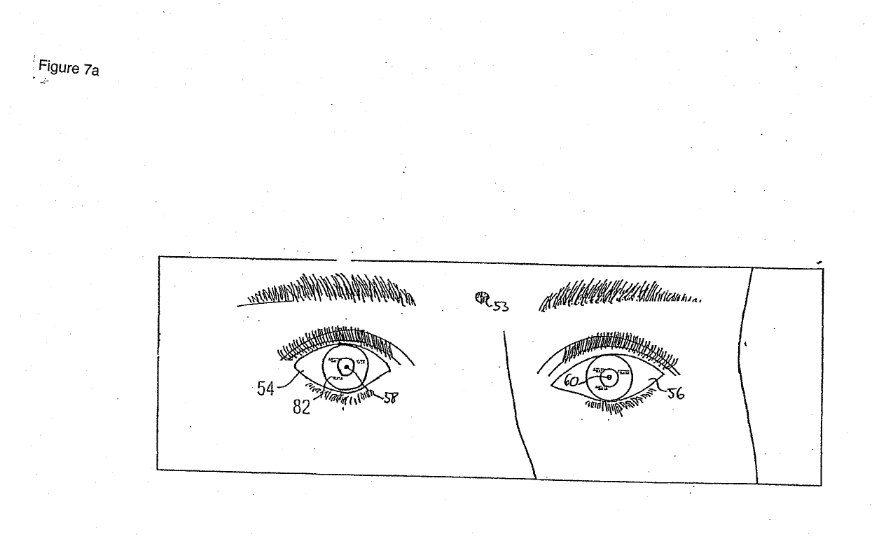

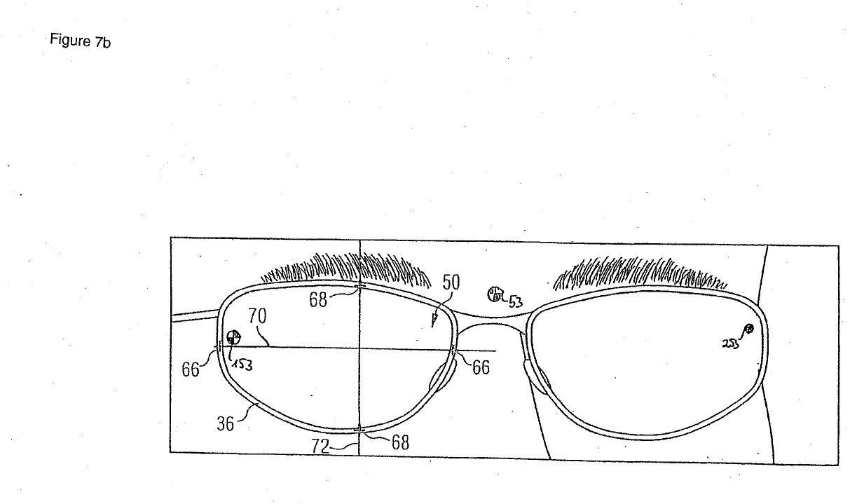

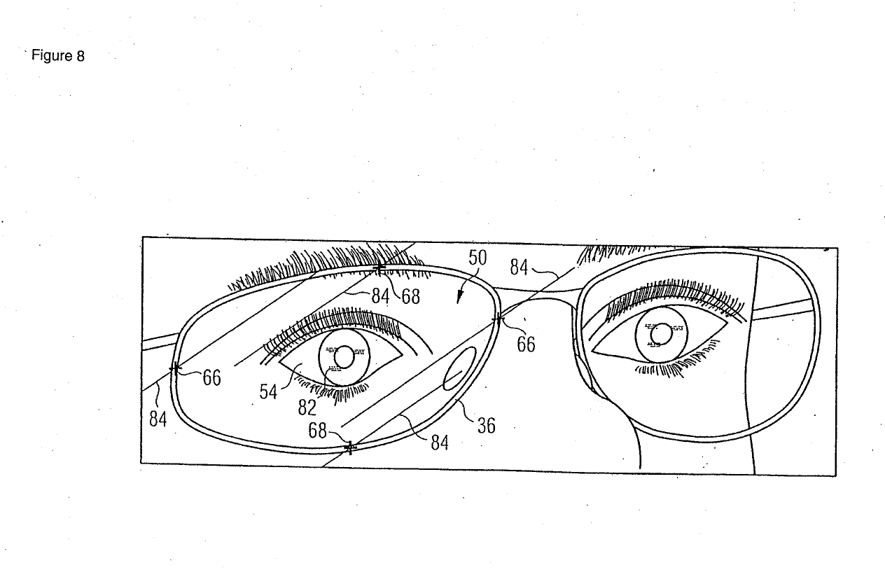

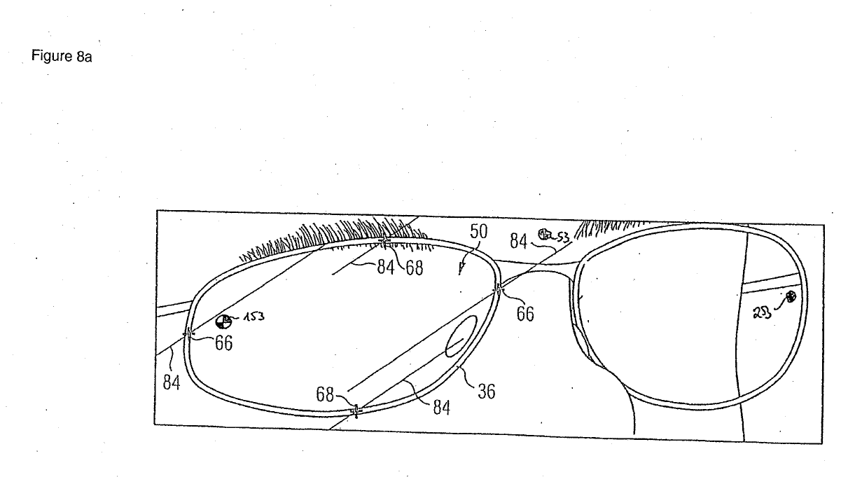

FIGS. 7 and 8 show images that are generated by the upper camera 16 (FIG. 7) and the lateral camera 16 (FIG. 8). The images also show the intersection points 66, 68 of the horizontal plane 70 and the vertical plane 72 as well as the reflexes 82 for the right eye 54 of the user 30. FIG. 8 shows projections of the possible intersection points of the horizontal plane 70 and the vertical plane 72 with the rim 36 of the spectacle frame 52 as the straight lines 84, taking the perspective view of the lateral camera 16 into consideration.

FIG. 7a shows a schematic view of comparative image data as generated by the upper camera 14, i.e. a schematic front view of a subarea of the head of the user 30 without spectacles, wherein merely a right eye 54 and a left eye 56 of the user 30 are illustrated. A pupil center point 58 of the right eye 54 and a pupil center point 60 of the left eye 56 are shown as user data in FIG. 7. Furthermore, FIG. 7 shows the saddle point 53.

Preferably, the pupil center points 58, 60 and the saddle point 53 are determined automatically by a user data positioning device (not shown). To this end, reflexes 82 are used, which arise on the corneas of the respective eyes 54, 56 due to the illuminants 28. Since according to the embodiment of the apparatus 10 shown in FIG. 1, three illuminants 28 are arranged, for example, three reflexes 82 are imaged per eye 54, 56. The reflexes 82 arise for each eye 54, 56 directly at the penetration point of a respective illuminant visual axis on the cornea. The illuminant visual axis (not shown) is the straight connecting line between the location of the respective illuminant 28, which is centrally imaged on the retina, and the respective pupil center point 58, 60 of the corresponding eye 54, 56. The elongation of the illuminant visual axis (not shown) passes through the optical ocular center of rotation (not shown). Preferably, the illuminants 28 are arranged such that they lie on a conical cylindrical surface, the apex of the cone being located at the pupil center points 58 and 60 of the right eye 54 and the left eye 56, respectively. Starting from the cone apex, the axis of symmetry of the cone is arranged in parallel to the effective optical axis 20 of the upper camera 14, wherein the three illuminants 28 are further arranged such that straight connecting lines of the cone apex and the respective illuminant 28 merely intersect in the cone apex.

The pupil center points 58 and 60 of the right eye 54 and the left eye 56, respectively, can be determined on the basis of the reflexes 82 for the right eye 54 and the left eye 56, and in particular the position in the three-dimensional space of the saddle point 53 relative to the pupil center points 58 and 60 of the right eye 54 and the left eye 56, respectively.

FIGS. 7b and 8a shows images that are generated by the upper camera 16 (FIG. 7b) and the lateral camera 16 (FIG. 8a). The images also show the intersection points 66, 68 of the horizontal plane 70 and the vertical plane 72. FIG. 8a shows projections of the possible intersection points of the horizontal plane 70 and the vertical plane 72 with the rim 36 of the spectacle frame 52 as the straight lines 84, taking the perspective view of the lateral camera 16 into consideration.

Advantageously, according to the apparatus 10, the optical parameters, i.e. for example interpupillary distance, corneal vertex distance, face form angle, pantoscopic angle, and fitting height, can be determined for a user 30 whose exe excursion does not correspond to the zero direction of sight, and actual values of the fitted spectacles can be compared to predetermined values. Instead, the user 30 looks at the image of the bridge of his nose in the partially transparent minor 26 at a distance of approximately 50 to 75 cm according to the preferred embodiments. In other words, the user 30 is located at a distance of approximately 50 to approximately 75 cm in front of the partially transparent mirror 26, and looks at the image of his face in the partially transparent minor 26, in particular at the root of his nose. The position of the eyes 54, 56 resulting from the object looked at, i.e. the convergence of the eyes 54, 56, may be taken into account in the determination of the optical parameters, and rotations of the eyes can e.g. be compensated for when determining the optical parameters, wherein for example a virtual zero direction of sight can be determined considering the actual eye excursion, and the optical parameters of the user can be determined on the basis of the virtual zero direction of sight, i.e. the determined and unmeasured zero direction of sight. Advantageously, the distance between the user 30 and the cameras 14, 16 can thus be small. In particular, it is also possible to approximately predetermine the optical parameters. Furthermore, the spectacles 38 may be prefitted and the optical parameters may be determined using the apparatus 10 for the prefitted spectacles.

Moreover, according to a further preferred embodiment, the apparatus 10 is designed to calculate the pantoscopic angle of the spectacles 38 for each spectacle lens from the angle between the straight line through the upper intersection point 68 and the lower intersection point 68 of the vertical intersection plane 72 with the rim 36 of the spectacle frame 52 in the three-dimensional space. In addition, a mean pantoscopic angle can be determined from the pantoscopic angle determined for the right eye 54 and the pantoscopic angle determined for the left eye 56. Furthermore, a warning notification may be output if the pantoscopic angle of the right spectacle lens deviates from the pantoscopic angle of the left spectacle lens by at least a predetermined maximum value. Such a notification may be output by means of the monitor 18, for example. Analogously, the face form angle and the corneal vertex distance or the interpupillary distance may be determined from the three-dimensional data set for the right eye 54 and the left eye 56 as well as mean values thereof, and notifications may optionally be output via the monitor 18 if the deviations of the values for the right eye 54 and the left eye 56 each exceed a maximum value.

The corneal vertex distance can selectively be determined according to reference point requirement or according to ocular center of rotation requirement. According to the reference point requirement, the corneal vertex distance corresponds to the distance of the vertex of the spectacle lens 50 from the cornea at the penetration point of the visual axis of the eye in the zero direction of sight. According to the ocular center of rotation requirement, the corneal vertex distance corresponds to the minimum distance of the cornea from the spectacle lens 50.

Furthermore, the apparatus 10 can be designed such that the fitting height of the spectacle lens 50 is calculated on the basis of a distance of the penetration point of the visual axis of an eye 54, 56 in the primary position with a lens plane of a spectacle lens 50 from a lower horizontal tangent in the lens plane. A lower horizontal tangent is e.g. the line 84 of the delimitation 62, 64 according to the boxing system in FIGS. 5b and 6b. Preferably, the apparatus 10 is designed such that a three-dimensional closed polyline is determined for the lens shape of the spectacle lens 50 from points on the rim 36 of the spectacle frame 52 for each eye 54, 56, wherein an averaged polyline for the lens shape can be determined from polylines of the respective spectacle lenses 50 of the right eye 54 and the left eye 56.

Alternatively, it is also possible that instead of averaging the values of the optical parameters, which are determined for the right eye 54 and the left eye 56, the optical parameters or the polylines for the lens shape are merely determined for the spectacle lens 50 of one of the eyes 54, 56, and these values are also used for the other of the eyes 54, 56.

Furthermore, the apparatus according to a preferred embodiment can be used to generate images of the user 30 and to superimpose image data of a multitude of frame and/or spectacle lens data on these images, whereby it is possible to advise the user 30 optimally. In particular, materials, layers, thickness, and colors of the spectacle lenses, the image data of which are superimposed on the generated image data, can be varied. Therefore, the apparatus 10 can be designed to provide fitting recommendations, in particular optimized individual parameters, for a multitude of different spectacle frames or spectacle lenses.

In particular, the apparatus is designed to determine the above parameters and values for produced spectacles using at least one saddle point 53, and to compare them to corresponding predetermined parameters and values. In particular, the actual position of wear of the spectacles can be compared to a predetermined position of wear, according to which the spectacles have been produced, and deviations from the predetermined position of wear can be corrected. Here, the predetermined parameters can be stored by the apparatus and retrieved from the memory thereof. The predetermined parameters and values may also be supplied to the apparatus.

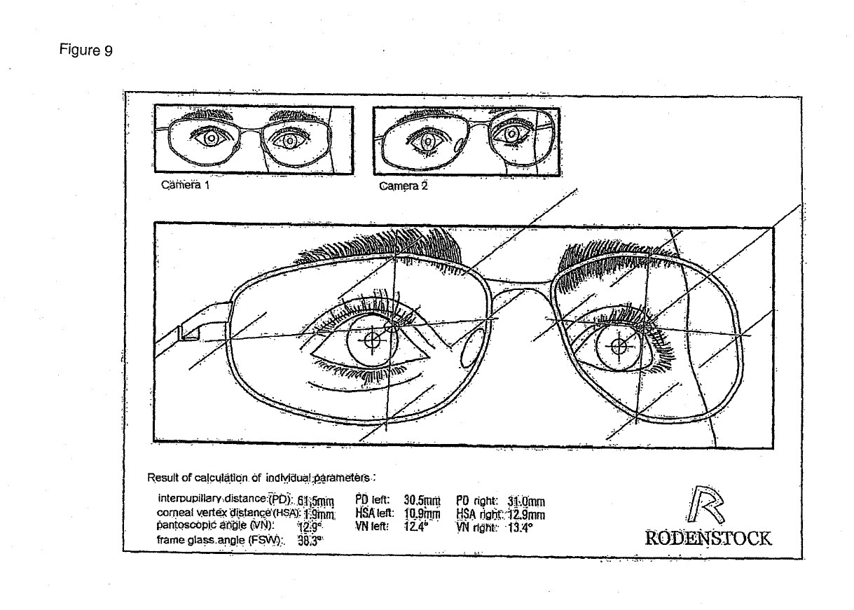

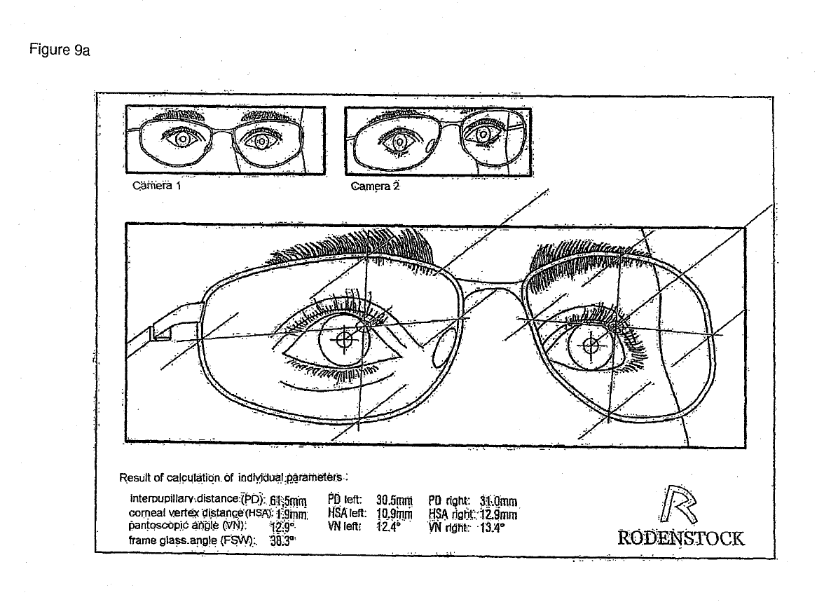

FIG. 9 shows an output image as may be displayed on the monitor 18, the image data of the upper camera 14 (referred to as camera 1) and the lateral camera 16 (referred to as camera 2) being illustrated. Furthermore, an image of the lateral camera 16 is shown on which the user data are superimposed. Furthermore, the optical parameters for the right eye 54 and the left eye 56 well as mean values thereof, are illustrated.

Preferably, multiple illuminants 28 are arranged such that for all cameras 14, 16 reflexes 82 for each eye 54, 56 are generated directly at the penetration point of the respective visual axis on the cornea or geometrically defined around the penetration point. Furthermore, the illuminants 28 are preferably arranged such that the reflexes 82 are in particular generated for the penetration point of the respective visual axis of the eyes 54, 56 in the primary position. Particularly preferably, for both eyes, approximately geometrically defined corneal reflexes are arranged around the penetration point for the upper camera 14 and, for the lateral camera 16, reflexes are arranged at the penetration points of the visual axes of the eyes 54, 56 in the primary position, by an illuminant 28 on the effective optical axis 22 of the lateral camera 16 reflected on the respective center parallel line of the two visual axes of the eyes 54, 56 in the primary position, and two further illuminants 28, which are arranged on the cone, which is defined as the cone axis by the central parallel line of the visual axes of the eyes 54, 56 in the primary position and as the generatrix by the effective optical axis 20 of the lateral camera 16, such that all illuminants 28 lie on disjunctive generatrices of the cone and the employed illuminants 28 have horizontal extensions that satisfy the equation

(mean interpupillary distance)/(horizontal extension)=(distance of upper camera 14 to eye 54, 56)/(distance of illuminant 28 to eye 54, 56).

FIG. 9a shows an output image according to FIG. 9. The illustrated output image is a superimposition of the image data with the comparative image data.

By means of the above-described embodiment, it is further possible to check or determine the position of spectacles of the first and/or the second spectacle lens in the position of wear relative to the eyes or the pupils of the user in a simple manner. In particular, it is thus possible to determine an actual position of wear of spectacles having individually fitted spectacle lenses and to compare it with a desired target position of wear used for the individual fitting of the spectacle lenses. If the actual position of wear deviates from the target position of wear, in particular the position of the spectacles or of the first and/or second spectacle lens in the actual position of wear can be corrected such that the actual position of wear corresponds to the desired target position of wear. The target position of wear is the position of wear of the spectacles on the basis of which the individually fitted spectacle lenses are produced. When checking the actual position of wear, the actual centration of a spectacle lens or of both spectacle lenses in the spectacle frame, i.e. the position of a spectacle lens relative to the spectacle frame, can advantageously be ascertained and checked and be taken into consideration in the determination and correction of the actual position of wear.

In other words, the desired target position of wear of spectacles to be produced can be determined as well by means of the above-described apparatus in a simple manner. The spectacles to be produced with individual spectacle lenses can be produced in the following taking the desired target position of wear into consideration. If the spectacles produced according to target position of wear are used, it is possible, however, that the actual position of wear of the spectacles, i.e. in particular of the two spectacle lenses, thus the actual position of the spectacles or the spectacle lenses relative to the corresponding eyes of the user, deviates from the target position of wear. To correct such deviations, it may therefore be necessary to adjust the spectacle frame after the production of the spectacles such that the actual position of wear corresponds to the prior determined, desired target position of wear. This adjustment can be performed by an optician, for example.

To this end, first of all comparative image data of at least subareas of the head of the user are generated, the user not wearing the already produced spectacles though. Auxiliary marks or auxiliary points, for example characteristic features of the subarea of the head, are determined in the comparative image data. The auxiliary points may be special features of the subarea of the head of the user, such as a birthmark, scars, light or dark pigmentation marks, etc. The auxiliary points may also be artificially produced points, e.g. so-called saddle points, attached to predetermined or predeterminable positions of the subarea of the head in the form of adhesive labels. An exemplary saddle point 53 is illustrated in FIG. 5b.

In particular, the auxiliary points 53 are chosen at positions of the subarea of the head or the saddle points 53 are arranged accordingly, so that the saddle points 53 are spatially constant or unchangeable relative to the respective ocular centers of rotation.

Furthermore, in addition to the auxiliary points, also the pupil positions or pupil center points of the user, preferably in the zero direction of sight of the user, are determined in the image data of the subarea of the head as well. The spatial locations of the pupil center points are further determined relative to the auxiliary points.

Subsequently, image data of the subarea of the head of the user are generated, wherein the user wears the produced spectacles 38 with the individually manufactured spectacle lenses in the actual position of wear.

In doing so, a further saddle point 153, 253 is arranged or drawn on a spectacle lens or on both spectacle lenses, which allow determining e.g. the position of the engraved points and in particular determining the position of the engraved points in the box dimension of the corresponding spectacle lens. Consequently, the saddle point illustrated in 5b may also present a presenting means 153, 253. For example, the presenting means 153, 253 may be formed as an adhesive label 153, 253. However, the presenting means 153, 253 may also be a single-color point 153, 253 which can be arranged on the spectacle lens (shown in FIG. 6a) either as an adhesive label or which is drawn directly onto the spectacle lens (shown in FIG. 6a) e.g. with a pencil.

Parameters of the spectacles or the first and/or the second spectacle lens relative to the auxiliary points are determined using the above-described image data. Since now both the relative positions of the pupil centers 58, 60 with respect to the auxiliary points 53 and the relative position of the spectacles 38 or the first and/or the second spectacle lens in their actual positions of wear with respect to the auxiliary points are known, the actual position of the spectacles 38 relative to the pupil centers 58, 60 can be determined in a simple manner, for example by means of a coordinate transformation. Therefore, it is possible to identify a deviation of the actual position of wear from the target position of wear and to compensate for it afterwards. For example, the actual corneal vertex distance can be determined and compared to the corneal vertex distance taken into account for the calculation and production of the individual spectacle lenses 50. If the two parameters do not match, the spectacles 38 can be adjusted further, i.e. the actual position of wear can be varied and the new actual position of wear can be checked with the above-described method. Alternatively, the actual position of wear can be determined again, compared to the target position of wear, and varied or adjusted until the deviation of the actual position of wear from the target position of wear is smaller than an acceptable, predetermined deviation threshold. In doing so, the actual location of each spectacle lens can be taken into account due to the centration data determined by means of the presenting means.

Furthermore, the correction of the actual position of wear cannot be performed on the basis of the corneal vertex distance. Instead, the actual position of wear can be adjusted further to the target position of wear with respect to further or other individual parameters.

Advantageously, the actual position of wear can therefore be adjusted to the target position of wear in a simple manner even if the individually produced spectacle lenses 50 are already arranged in the spectacles 38, and optionally a faulty arrangement of the spectacle lenses in the spectacle frame can be corrected. Measuring errors in the determination of the actual position of wear are thereby avoided or are very few, since the positions of the pupil centers 58, 60 relative to the spectacles 38 or relative to the first and/or the second spectacle lens are not determined through the spectacle lenses 50, but by means of the auxiliary points. For example, a faulty determination of the position of the spectacles 38 or of the first and/or the second spectacle lens relative to the pupil centers 58, 60, which may occur due to the optical properties of the spectacle lenses 50, is avoided. The position of the auxiliary points 53 relative to the pupil centers 58, 60, however, was determined in the absence of the spectacles 38 or of the first and/or the second spectacle lens, which is why no measurement is performed through the spectacle lenses 50 either in this case.

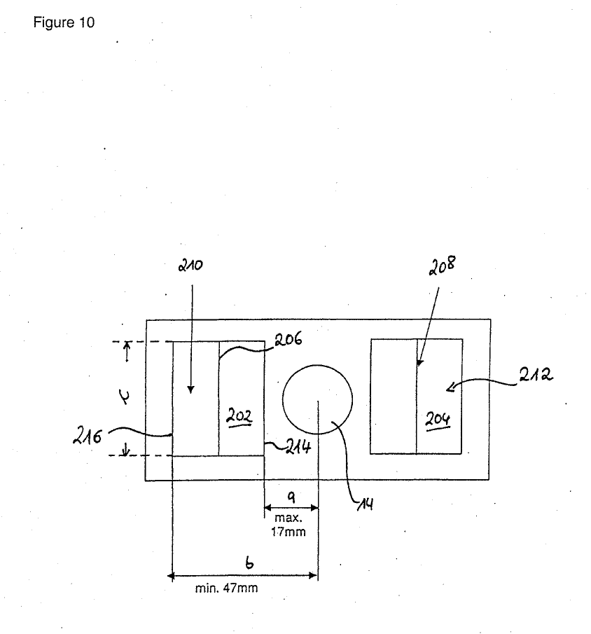

FIG. 10 shows a front view of a section of the apparatus 10 as shown in FIG. 1. In particular, FIG. 10 shows a first fixation target 202 and a second fixation target 204. A camera 14 is arranged between the two fixation targets 202, 204. As shown in FIG. 1, the two fixation targets 202, 204 may be arranged laterally next to the mirror 26. The two fixation targets 202, 204 may also be arranged behind the mirror 26. In this case, it is sufficient for the mirror 26 to be transparent at least in the spectral region of fixation lines 206, 208 such that the fixation line 206 or the fixation line 208 is visible as a preferred light field through the partially transparent minor 26. The presenting element of the fixation target 202 is a cylinder lens 210. The presenting element of the fixation target 204 is a cylinder lens 212. The camera 14 shown in FIG. 10 comprises a camera lens with an opening having a diameter of approximately 30 mm. In this case, the maximum distance a of the center of the opening of the camera lens of the camera 14 and a lateral rim 214 opposite to the camera 14 is approximately 17 mm. The remaining rim 216 of the cylinder lens 210 is distanced from the center of the opening of the camera lens of the camera 14 with a distance b of at least approximately 47 mm. Analogous explanations apply to the camera 14 and the cylinder lens 212.

In this exemplary illustration, the visible area of the cylinder lens has a height of approximately 40 mm, i.e. the cylinder lens has a height c of at least approximately 40 mm. Consequently, also the fixation line 206 is at least 40 mm in length. The same applies to the cylinder lens 212 and the fixation line 208.

Preferably, the cylinder lenses 210, 212 are aligned such that a cylinder axis (not shown) of the respective cylinder lenses 210, 212 is arranged substantially vertically in the frame of reference of the earth. Due to the light source (shown in the following figures) being arranged substantially in the focal plane or focal line of the cylinder lens, the fixation lines 206, 208 are generated by light that is substantially diffused substantially along the vertical direction (in the frame of reference of the earth) and substantially parallel substantially along the horizontal direction (in the frame of reference of the earth). In other words, when the test person (30 shown in FIG. 1) looks at the cylinder lenses 210, 212, he can see the fixation lines 206, 208, wherein if the test person looks at the fixation lines 206, 208, he is free to choose the head posture in the vertical direction. Consequently, the test person will choose the head posture according to his natural head posture. Since the light in the horizontal plane is substantially parallel, the fixation lines 206, 208 appear to be imaged to infinity for the test person. Consequently, it is made possible by means of the apparatus shown in FIG. 10 that the test person assumes his habitual head and body posture with his view to infinity. In this position, the individual parameters can be determined, for example.

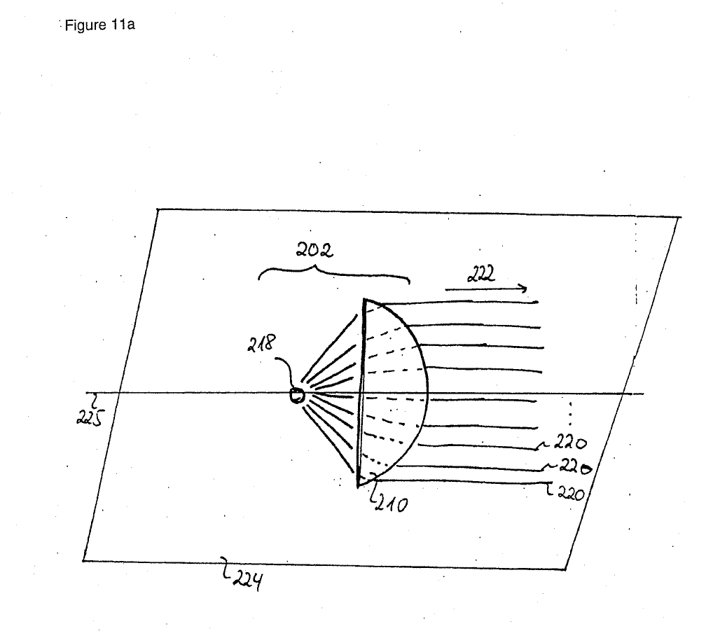

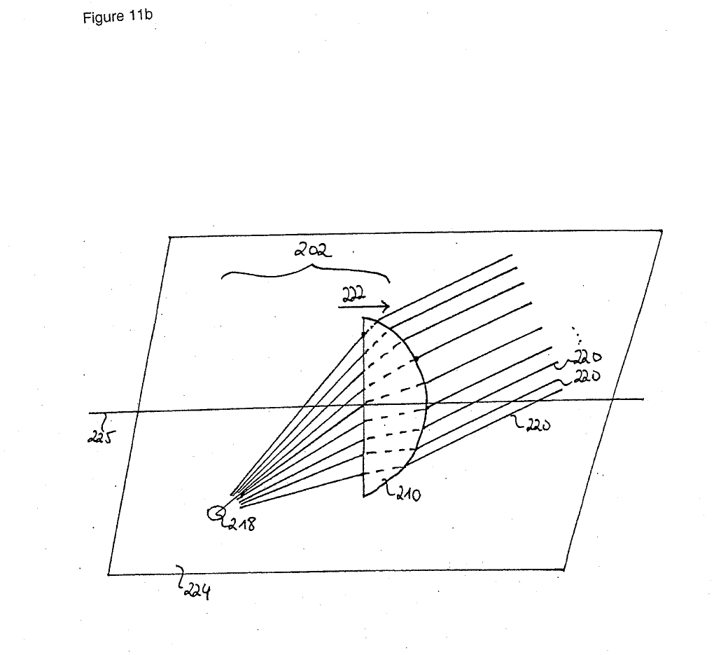

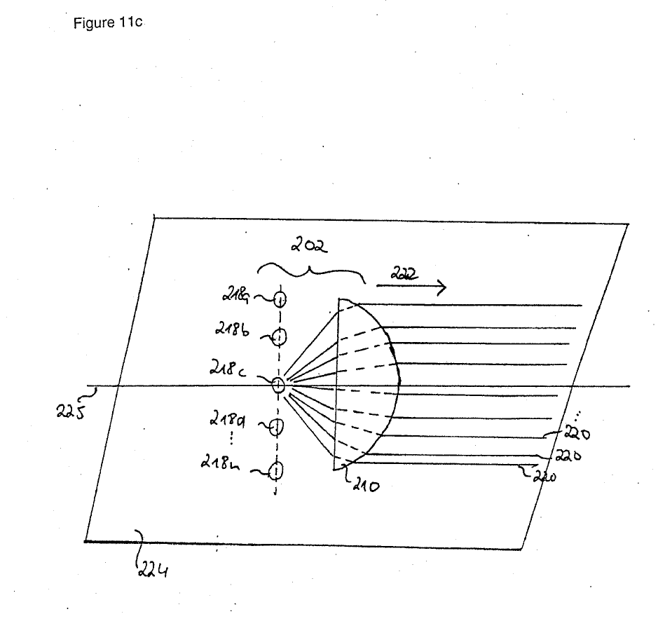

FIG. 11a shows a schematic top view of the fixation target 202. The fixation target 202 comprises the cylinder lens 210 and an illuminating device 218. The illuminating device 218 shown in FIG. 11a may comprise an LED, in particular a homogenous LED, an incandescent lamp, or a similar light source. It is also possible for the illuminating device 218 to comprise a ground glass (not shown). The illuminating device 218, in particular the light source thereof, as is shown in FIG. 11a, is substantially arranged on a focal line of the cylinder lens 210. Consequently, the electromagnetic radiation 220, which passes through the cylinder lens 210 starting from the illuminating device 218, is substantially parallel. If the cylinder axis, i.e. the focal line of the cylinder lens 210, is arranged substantially vertically, the electromagnetic rays 220 are substantially located in a horizontal plane in the frame of reference of the earth. An optical axis of the fixation target 202 is an axis that is substantially parallel to the electromagnetic radiation 120. The optical axis is drawn in as an arrow 222. The horizontal plane 224 is drawn in likewise.

Furthermore, a vertical plane 225 is shown in FIG. 11a. The vertical plane 225 is shown in the form of a line due to the top view of FIG. 11a. The intersection line between the vertical plane 225 and the horizontal plane 224 is preferably parallel to the optical axis 222. The optical axis 222 is preferably parallel to a horizontal direction in the frame of reference of the earth. It is also possible for the vertical plane 225 and the horizontal plane 224 to be arranged vertically and horizontally, respectively, with respect to a frame of reference deviating from the frame of reference of the earth.

FIG. 11b shows a view of the fixation target 202 according to FIG. 11a, wherein the illuminating device 218 does not comprise the focal line of the cylinder lens 210. However, the illuminating device 218 is arranged in the focal plane of the cylinder lens 210. Thus, the electromagnetic radiation 220 is parallel to each other after passing through the cylinder lens 210, but not parallel to the optical axis 222. If the illuminating device 218 is arranged such that a light-emitting surface of the illuminating device is arranged in the focal plane and is substantially parallel to the focal line of the cylinder lens 210, the electromagnetic radiation is parallel in each horizontal plane 224a, 224b, 224c, . . . after passing through the cylinder lens 210, wherein the direction of the parallel electromagnetic radiation is substantially identical for all horizontal planes 224a, 224b, 224c, . . . .

FIG. 11c shows a view of a fixation target 202 similar to that shown in FIG. 11a. However, the fixation target 202 comprises multiple illuminating devices 218a, 218b, 218c, . . . , 218n. 5 illuminating devices are exemplarily illustrated. The illuminating device 218c comprises the focal line of the cylinder lens 210. After passing through the cylinder lens, the electromagnetic radiation 220 of the illuminating device 218c is parallel to each other and parallel to the optical axis 222. The electromagnetic radiation of the further illuminating devices 218a, 218b, 218c, 218d, . . . , 218n is not drawn in. As an example, the illuminating device 218d is arranged similar to the illuminating device 218 illustrated in FIG. 11b, which is why the beam path (not shown) of the electromagnetic radiation starting from the illuminating device 218d is similar to that shown in FIG. 11b. Preferably, all illuminating devices 218a, 218b, 218c, 218d, . . . , 218n are arranged in the focal plane of the cylinder lens 210 or comprise the focal plane of the cylinder lens 210 at least partially.

Every light field can be generated by corresponding different illuminating devices 218a, 218b, 218c, 218d, . . . , 218n, in particular substantially line-shaped luminous surfaces, which are located in the focal plane of the common cylinder lens 210. Due to the different lateral distances from the focal line, the different directions of the light field result (as shown in FIGS. 11a and 11b, wherein the light is always parallel in one direction).

Preferably, the illuminating devices 218a, 218b, 218c, 218d, . . . , 218n can be designed in a switchable manner, so that the direction of the light field can be changed by switching by only one illuminating device 218a, 218b, 218c, 218d, . . . , 218n being operated at a time. Thus, the direction of sight of the test person can be controlled, as preferably the light fields generated by the illuminating devices 218a, 218b, 218c, 218d, . . . , 218n are parallel to different directions and thus the test person has to look in different directions in order to be able to look at the light fields generated one after the other.

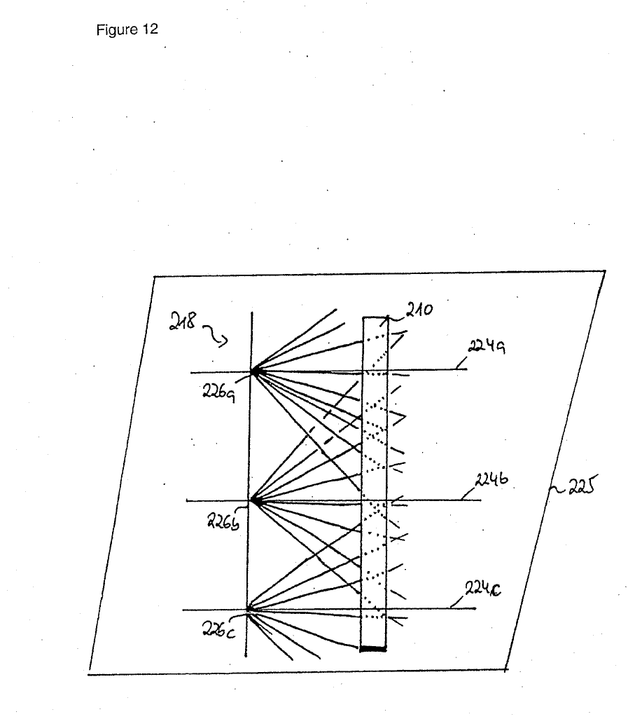

FIG. 12 shows a lateral sectional top view of the fixation target illustrated in FIG. 11a. In particular, FIG. 11a schematically illustrates the beam path at three exemplary points 226a, 226b, 226c of the illuminating device 218. The three exemplary points 226a, 226b, 226c are arranged in a vertical direction 228 one below the other. The vertical direction 228 is in particular a vertical direction in the frame of reference of the earth. Likewise, FIG. 12 shows three horizontal planes 224a, 224b, 224c. For example, electromagnetic radiation, which is radiated from the exemplary point 226a substantially in the horizontal plane 224a, is only substantially parallel after passing through the cylinder lens 210, as shown in FIG. 11a. In other words, FIG. 11a is a sectional view according to one of the planes 224a, 224b, 224c. Consequently, test person looking at electromagnetic radiation after passing through the cylinder lens 210 substantially sees diffused electromagnetic radiation along the vertical direction 228, whereas the one propagating in the planes 224a, 224b, 224c is substantially parallel to the optical axis 222.

In particular, the number and position of the exemplary points 226a, 226b, 226c is selected such that the electromagnetic radiation is substantially homogenous along the vertical direction 228 after passing through the cylinder lens 210. In other words, FIG. 12 exemplarily shows three points 226a, 226b, 226c. However, the above explanations apply to a large number of points, in particular to an infinite number of points of the illuminating device 218. The illuminating device 218 may comprise one or more diffuser(s) (not shown).

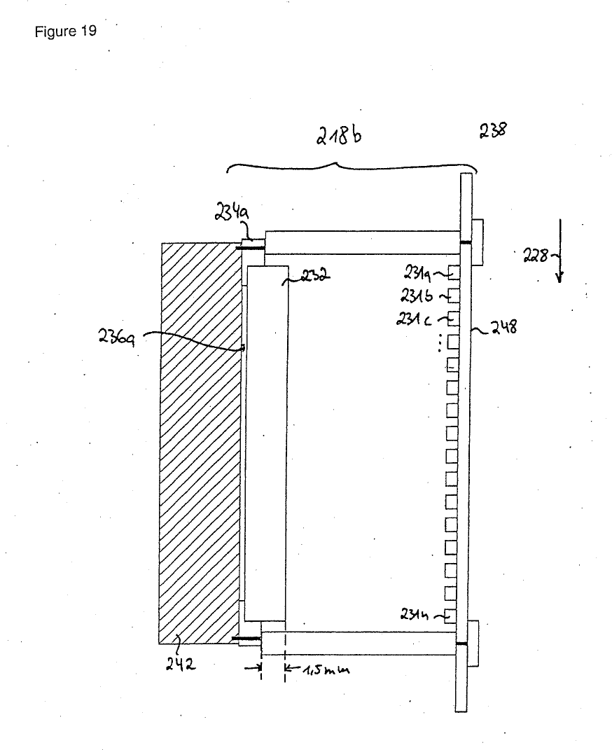

The illuminating device 218 may comprise one or more, in particular 16 light sources and a diffuser (see FIG. 19), wherein the light sources irradiate the diffuser and the diffuser comprises the points 226a, 226b, 226c, from which the electromagnetic radiation impinges on the cylinder lens 210.

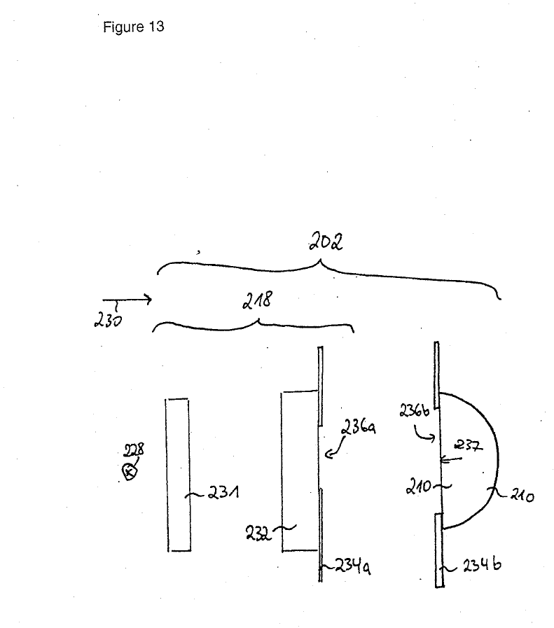

FIG. 13 shows a further schematic top view of a fixation target 202. The fixation target 202 comprises the cylinder lens 210 and the illuminating device 218. The illuminating device 218 comprises the light source 231, a diffuser 232, and an aperture diaphragm 234a. Also, the vertical direction 228 and the horizontal direction 230 are drawn in FIG. 13. Light, i.e. electromagnetic radiation, can exit from the light source 231 and irradiate the diffuser 232. The diffuser 232 causes the cylinder lens 210 to be irradiated substantially homogenously along the vertical direction 228. The aperture diaphragm 234a enables the restriction of electromagnetic radiation in particular substantially to a focal line (not shown) of the cylinder lens. To this end, the aperture diaphragm 234a may be variably adjustable, for example. It is also possible for the aperture diaphragm 234a to have a fixed size, in particular a diaphragm opening 236a of merely a few millimeters, for example smaller than 1.5 mm, smaller than 1 mm, smaller than 0.5 mm, smaller than 0.1 mm, smaller than 0.05 mm±0.02 mm in width. The aperture diaphragm is at least greater than 0.05 mm, greater than approximately 0.1 mm±0.02 mm in width. Furthermore, FIG. 13 shows an aperture diaphragm 234b. The aperture diaphragm 234b has a diaphragm opening 236b. The aperture diaphragm 234b is preferably formed and arranged such that a back surface 237 of the cylinder lens is not irradiated completely with electromagnetic radiation of the illuminating device 218, but mere a part of the back surface 237. Thus, the illuminated region of the cylinder lens 210 is limited, so that advantageously unfavorable effects occurring at the rim of the cylinder lens 210, such as refraction and diffusion, can be avoided. For example, the diaphragm opening 236b may have a width of approximately 70%, approximately 80%, approximately 90% of the width of the back surface 237 of the cylinder lens 210. In FIG. 13, the longitudinal direction of the cylinder lens 210 is substantially along the vertical direction 228 and the widthwise direction is substantially perpendicular to the vertical direction 228.

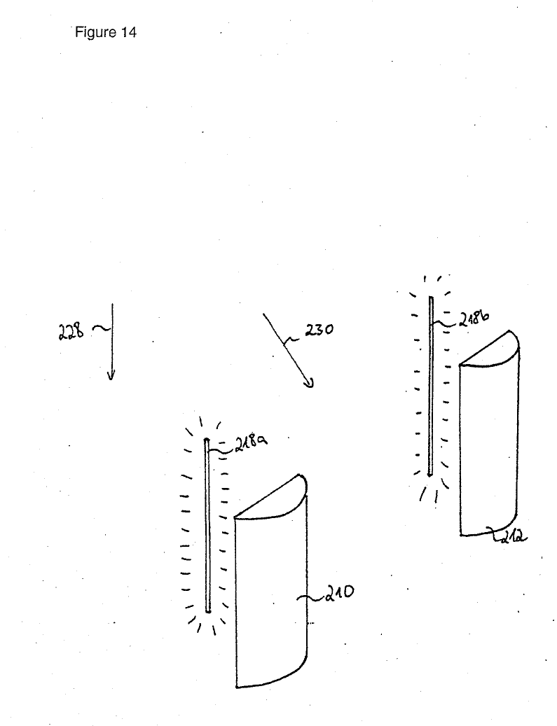

FIG. 14 shows a left cylinder lens 210 and a right cylinder lens 212. An illuminating device 218a is shown in the horizontal direction 230 behind the left cylinder lens 210. An illuminating device 218b is drawn in along the horizontal direction 230 behind the second cylinder lens 212. The illuminating devices 218a, 218b, which may be formed as light strips, are longitudinally extended along the vertical direction 228. In particular, the illuminating devices 218a, 218b radiate substantially homogenous light, i.e. substantially electromagnetic radiation of identical wavelength, along the vertical direction 228. After passing through the cylinder lenses 210, 212, the electromagnetic radiation is still diffused in the vertical direction 228. Electromagnetic radiation, which passes through the cylinder lenses 210, 212 in parallel to a horizontal plane (not shown), is substantially parallel to the horizontal direction 230. The illuminating devices 218a, 218b may be formed like in FIG. 13. The illuminating devices 218a, 218b may also each comprise 1, 2, 3, 5, 10, etc., homogenous LEDs, which are arranged one below the other along the vertical direction 218, for example, wherein the homogenous LEDs of the illuminating device 218a are arranged such that they generate a uniform, common light field that is substantially homogenous. This applies to the illuminating device 218b analogously.

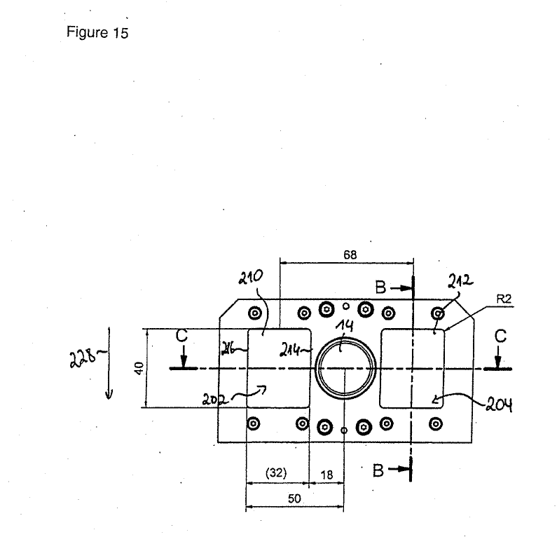

FIG. 15 shows a further schematic sectional view of a front view of a region of the apparatus 10, comprising a first fixation target 202 and a second fixation target 204. The fixation targets 202 and 204 comprise a cylinder lens 210 and 212, respectively. Also, a camera lens of a camera 14 is shown. The geometric centers of the fixation targets 202, 204 are distanced from each other approximately 68 mm, for example. The vertical dimension of the fixation targets 202, 204 is approximately 40 mm. The horizontal dimension of the fixation targets 202, 204 is approximately 32 mm. The distance of the rim 214 from a center of the camera lens of the camera 14 is approximately 18 mm. The distance of the rim 216 from the cylinder lens 210 is approximately 50 mm from the center of the camera lens of the camera 14. FIG. 15 is an engineering drawing, preferred measures being indicated in FIG. 15.

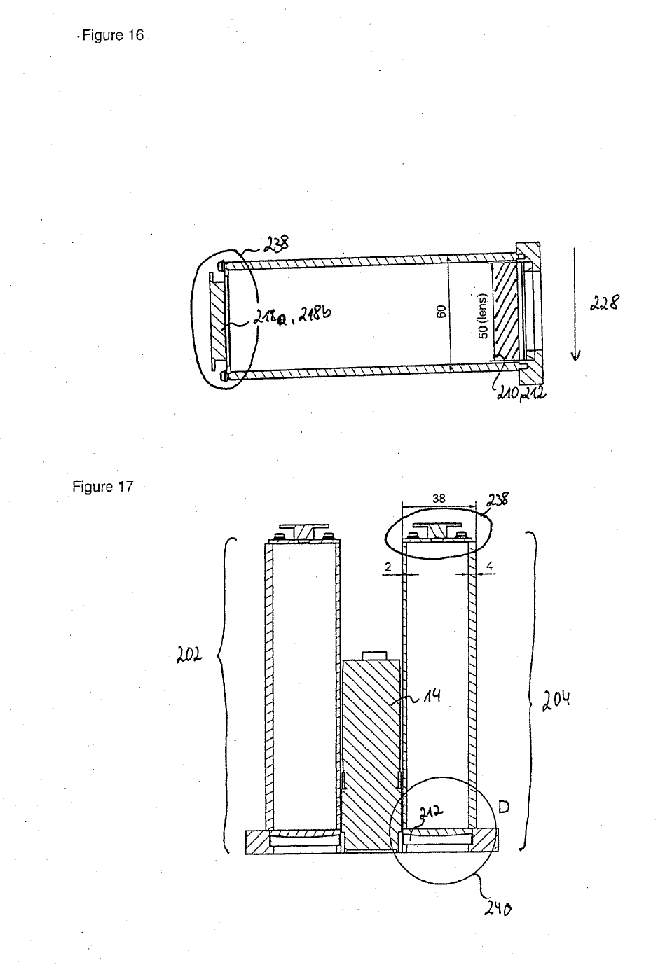

FIG. 16 shows a sectional view along the sectional plane BB, as shown in FIG. 15. Thus, FIG. 16 shows a lateral sectional of a fixation target, for example of the fixation targets 202 or 204. The fixation target 202, 204 has an extension of approximately 60 mm along the vertical direction (outer distance), wherein the schematically drawn cylinder lens 201, 212 has an extension of approximately 50 mm along the vertical direction. Furthermore, FIG. 16 shows a region 238, which is exemplarily illustrated in FIG. 19 in an enlarged manner. In the region 238, the illuminating device 218a, 218b is arranged in particular.

FIG. 17 shows a sectional view along the plane CC, as shown in FIG. 15.

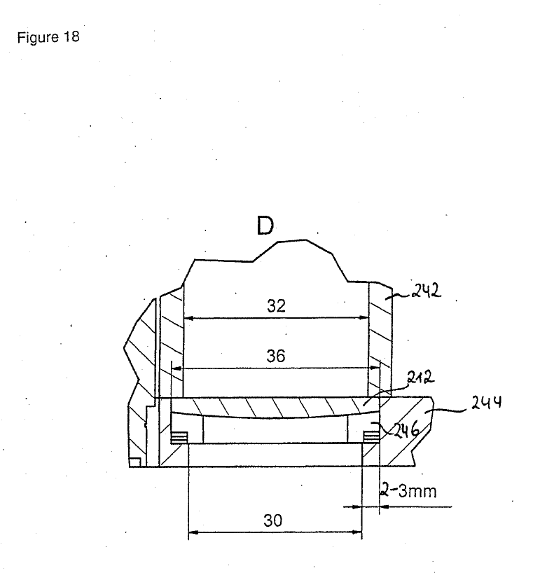

Two fixation targets 202, 204 as well as the camera 14 and the housing thereof are shown. The fixation target 204 has the illuminating device 218b in the rear region 238 (see FIG. 19). The same applies to the fixation target 202, wherein this has not been emphasized. The fixation target 204 has a width of approximately 38 mm, wherein the wall thicknesses of the two walls are approximately 2 mm and 4 mm. The fixation target 204 has a cylinder lens 212 in the front region 240. This region is illustrated in FIG. 18 in an enlarged manner.

FIG. 18 shows an enlarged view of the region 240. FIG. 18 illustrates the cylinder lens 212 and the profile 242 of the fixation target 212. Moreover, a wall 244 in the form of an L angle is illustrated, in which the cylinder lens 212 is arranged. For example, the cylinder lens 212 can be fixed by means of rubber 246. The wall 244 may be a component of the apparatus 10. However, it may also be a component of the fixation target 212 independent from the apparatus, so that e.g. the fixation target 212 can be taken out from the apparatus 10 in particular together with the fixation target 210. In this sectional view, the profile 242 of the fixation target 204 has an inner diameter of approximately 32 mm.

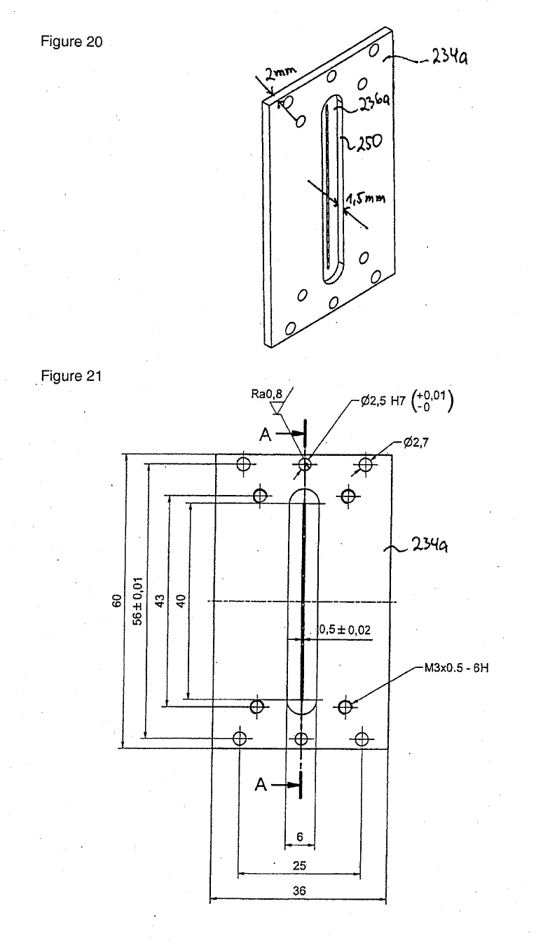

FIG. 19 shows an enlarged illustration of the illuminating device 218b as arranged in the rear region 238 of the fixation target 204. In FIG. 19, a multitude of light sources 231a, 231b, 231c, . . . , 231n is arranged at a rear end, in particular at a rear wall 248. In particular, 16 light sources may be arranged. The light sources may be LEDs, in particular single-color or multi-color LEDs, for example. The light sources 231a, . . . , 231n may also be conventional incandescent lamps, neon lamps, etc. In particular, instead of the 16 light sources 231a, . . . , 231n, merely one extended light source, for example a neon lamp, may be arranged. The light sources 231a, . . . , 231n illuminate a diffuser 232. The diffuser 232 may be a Plexiglas sheet with a thickness of approximately 3 mm, wherein a diaphragm 234a may be arranged on the diffuser 232. An exemplary diaphragm is shown in FIGS. 20, 21. In particular, the diaphragm has a diaphragm opening 236a in the form of a slit having a vertical extension of approximately 40 mm, for example. Furthermore, FIG. 19 shows the profile 242 of the fixation target 204.

The face or side of the diffuser 232 facing the light sources 231a, . . . , 231n may have a distance of approximately 7.7 mm from the light sources 231a, . . . , 231n. In particular, the distance is selected such that the diffuser is illuminated as uniformly as possible. The diffuser 232 is in particular designed to radiate homogenous light that is diffused in the vertical direction 128. As is shown in FIG. 19, the 16 light sources 231a, . . . , 231n are evenly distributed, wherein for example a distance from the light sources 231a, . . . , 231n may be approximately 2.5 mm, and the distance of a rim of the topmost LED 231a from an outer rim of the bottommost LED 231n is approximately 42 mm.

FIG. 20 shows a perspective view of an aperture diaphragm 234a. The aperture diaphragm 234a has a thickness of approximately 2 mm. Moreover, the aperture diaphragm 234a has an aperture opening 236a in the form of a slit. The aperture opening 236a is arranged in a recess 250 of the aperture diaphragm 234a. The recess 250 may have a height of approximately 1.5 mm, i.e. the slit 236a may have a thickness of approximately 0.5 mm.

FIG. 21 shows a schematic sectional view of the aperture diaphragm 234a. FIG. 21 is an engineering drawing of the aperture diaphragm 234a, preferred measures of the aperture diaphragm 234a being indicated in FIG. 21.

The above explanations in particular apply to the intended use of the apparatus 10.

While the foregoing has been described in conjunction with an exemplary embodiment, it is understood that the term “exemplary” is merely meant as an example, rather than the best or optimal. Accordingly, the disclosure herein is intended to cover alternatives, modifications and equivalents, which may be included within the spirit and scope of the disclosed apparatus and method.

Additionally, in the preceding detailed description, numerous specific details have been set forth in order to provide a thorough understanding of the present invention. However, it should be apparent to one of ordinary skill in the art that the present invention may be practiced without these specific details. In other instances, well-known methods, procedures, components, and circuits have not been described in detail so as not to unnecessarily obscure aspects of the disclosure herein.

1. A method for measuring at least one optical parameter of a test person, the method comprising:
generating a flat extensive light field by a fixation target to align the direction of sight of the test person when the test person looks at the light fields;
generating image data, by at least one image recording device, of at least one subarea of the head of the test person; and
determining the at least on optical parameter based on the generated image data.

2. The method according to claim 1, further comprising:
diffusing the electromagnetic radiation of the light field in a first predetermined plane, wherein
the electromagnetic radiation of the light field is substantially parallel in a second predetermined plane, which is substantially perpendicular to the first predetermined plane.

3. The method according to claim 1, wherein the fixation target comprises a cylinder lens, and wherein the first predetermined plane is substantially parallel to a cylinder axis of the cylinder lens, and the second predetermined plane is substantially perpendicular to the cylinder axis of the cylinder lens.

4. The method according to claim 3, further comprising arranging the cylinder axis in a substantially a vertical plane.

5. The method according to claim 1, further comprising forming the light field such that it is perceived as a line by the user.

6. The method according to claim 2, further comprising generating a substantially homogenously diffused light field, by an illuminating device of the fixation target, in a first direction that is substantially perpendicular to the second predetermined plane.

7. The method according to claim 6, further comprising:
arranging a luminous surface of the illuminating device substantially perpendicular to the first predetermined plane and substantially parallel to the second predetermined plane; and
emitting electromagnetic radiation of substantially identical intensity by the luminous surface.

8. The method according to claim 1, wherein the flat extensive light field is a substantially rectangular light field.

9. The method according to claim 2, further comprising positioning the fixation target such that the direction of emitted electromagnetic rays, which are substantially parallel to the second predetermined plane, are substantially perpendicular to a facial plane of the test person.

10. The method according to claim 1, wherein the light field has a length of at least approximately 40 mm.

11. The method according to claim 1, further comprising:
providing two fixation targets; and
arranging the two fixation targets such that each eye of the test person perceives exactly one of the two fixation targets.

12. The method according to claim 11, wherein the arranging step further comprises positioning the two fixation targets such that the test person can fuse the respective images of the two fixation targets.

13. The method according to claim 11, further comprising illuminating each of the two fixation targets such that the test person only sees one of the two fixation target at a time.

14. An apparatus for measuring at least one optical parameter of a test person wearing spectacles, the apparatus comprising:
at least one fixation target configured to generate a flat extensive light field to align the direction of sight of the test person when the test person looks at the light filed;
at least one image recording device configured to generate image data of at least one subarea of the test person; and
a data processing unit configured to determine the at least one optical parameter based on the generated image data.

15. The apparatus according to claim 14, wherein the at least one fixation target is further configured to diffuse the electromagnetic radiation of the light field in a first predetermined plane, wherein
the electromagnetic radiation of the light field is substantially parallel in a second predetermined plane, which is perpendicular to the first predetermined plane.

16. The apparatus according to claim 14, further comprising two fixation targets, wherein the at least one image recording device is positioned between the two fixation targets.

17. The apparatus according to claim 14, wherein the at least one fixation target further comprises at least one cylinder lens, wherein the cylinder lens is substantially parallel to the first predetermined plane and is substantially perpendicular to the second predetermined plane.

18. The apparatus according to claim 14, further comprising an illuminating device, which comprises a substantially rectangular light-emitting surface.

19. The apparatus according to claim 18, wherein the illuminating device comprises at least two light emitting diodes.

20. The apparatus according to claim 19, wherein the illuminating device further comprises at least one diffuser, and wherein the light emitting diodes illuminate the diffuser such that the diffuser emits electromagnetic radiation with substantially homogenous intensity.

21. The apparatus according to claim 18, wherein the rectangular light-emitting surface is at least partially arranged substantially in a focal plane of the cylinder lens.

22. The apparatus according claim 14, wherein the image recording device comprises an aperture that is distanced between approximately 5 mm and approximately 40 mm from the at least one fixation target.

23. The apparatus according to claim 14, further comprising:
at least one presenting means configured to present at least one characteristic point of a spectacle lens, wherein
the at least one image recording device is further configured to generate additional image data of the at least one presenting means and at least of subareas of a spectacle lens of the spectacles and a spectacle frame of the spectacles, and wherein
the data processing unit is further configured to determine a position of a spectacle lens relative to the spectacle frame based on the additional image data.

24. An apparatus for measuring at least one optical parameter of a test person wearing spectacles the apparatus comprising:
at least one fixation target configured to generate a flat extensive light field to align the direction of sight of the test person when the test person looks at the light field;
at least two image recording devices, each configured to generate image data of at least subareas of the head of the test person;
a data processing unit configured
to determine user data of at least the subarea of the head or at least the subarea of the head and spectacles, arranged on the head of the test person, in the position of wear of the test person on the basis of the generated image data, wherein the user data comprises location information in the three-dimensional space of predetermined points of the subarea of the head or the subarea of the head and spectacles;
a parameter determining device configured to determine the at least one optical parameter of the test person based on the user data; and
a data output device configured to output at least part of the determined at least one optical parameter.

25. The apparatus according to claim 14, further comprising:
at least two image recording devices, each configured to:
generate comparative image data of at least a subarea of the head of the test person in absence of the spectacles and/or in absence of the at least one spectacle lens and of at least a subarea of an auxiliary structure, and
generate image data of a substantially identical subarea of the head of the test person with spectacles arranged thereon and/or at least one spectacle lens arranged thereon and of at least the subarea of the auxiliary structure, wherein
the data processing unit is further configured to determine the position of the spectacles and/or of the at least one spectacle lens relative to the pupil center point of the corresponding eye of the test person in the zero direction of sight based on the image data, on the basis of the comparative image data and on the basis of the at least the subarea of the auxiliary structure.
